SEQUENCE LISTING (9 sequences)

SEQ ID NO: 1 - DNA, 2103 bp, Humicola grisea
atgcataccttctccaagctcctcgtcctgggctctgccgtccagtctgccctcgggcggcctcacggctcttcgcgtctccaggaacgcgctgccgttgataccttcatcaacaccgagaagcccatcgcatggaacaagctgctcgccaacatcggccctaacggcaaagccgctcccggtgccgccgccggcgttgtgattgccagcccttccaggacggaccctccttgtacgtggtggcatggaatggacccaagagactggttttagatgaaagagagtttctgctaaccgccacacccagacttcttcacctggacccgcgatgccgccctggtcctcaccggcatcatcgagtcccttggccacaactacaacaccaccctgcagaccgtcatccagaactacgtcgcgtcgcaggccaagctgcagcaggtctcgaacccctcgggaaccttcgccgacggctcgggtctcggtgaggccaagttcaatgtcgacctcactgccttcactggcgaatggggtcgccctcagagggacggcccgcccctgcgcgccatcgctctcatccagtacgccaagtggctgatcgccaacggctacaagagcacggccaagagcgtcgtctggcccgtcgtcaagaacgatctcgcctacacggcccagtactggaacgagaccggcttcgatctctgggaggaggtccccggcagctcgttctttaccatcgccagctctcacaggggtgagtcatttattgttcagtgttttctcattgaataattaccggaatgccactgacgccaaacagctctgactgagggtgcttacctcgccgctcagctcgacaccgagtgccgcgcctgcacgaccgtcgcccctcaggttctgtgcttccagcaggccttctggaactccaagggcaactatgtcgtctccaacagtaagatccctacaccaacaaaaaaaatcgaaaaggaacgttagctgacccttctagtcaacggcggcgagtatcgctccggcaaggacgccaactcgatcctggcgtccatccacaacttcgaccctgaggccggctgcgacaacctgaccttccagccctgcagcgagcgcgccctggccaaccacaaggcctatgtcgactcgttccgcaacctctacgccatcaacaagggcatcgcccagggcaaggccgttgccgtcggccgctactcggaggatgtctactacaacggcaacccgtggtacctggccaactttgccgccgccgagcagctctacgacgccatctacgtgtggaacaagcagggctccatcaccgtgacctcggtctccctgcccttcttccgcgaccttgtctcgtcggtcagcaccggcacctactccaagagcagctcgaccttcaccaacatcgtcaacgccgtcaaggcctacgccgacggcttcatcgaggtggcggccaagtacaccccgtccaacggcgcgctcgccgagcagtacgaccgcaacacgggcaagcccgactcggccgccgacctgacgtggtcgtactcggccttcctctcggccatcgaccgccgcgcgggtctcgtccccccgagctggcgggccagcgtggccaagagccagctgccgtccacctgctcgcgcatcgaggtcgccggcacctacgtcgccgccacgagcacctcgttcccgtccaagcagaccccgaacccctccgcggcgccctccccgtccccctacccgaccgcctgcgcggacgctagcgaggtgtacgtcaccttcaacgagcgcgtgtcgaccgcgtggggcgagaccatcaaggtggtgggcaacgtgccggcgctggggaactgggacacgtccaaggcggtgaccctgtcggccagcgggtacaagtcgaatgatcccctctggagcatcacggtgcccatcaaggcgacgggctcggccgtgcagtacaagtatatcaaggtcggcaccaacgggaagattacttgggagtcggaccccaacaggagcattaccctgcagacggcgtcgtctgcgggcaagtgcgccgcgcagacggtgaatgattcgtggcgttaa

SEQ ID NO: 2 - protein, 634 aa, Humicola grisea
MHTFSKLLVLGSAVQSALGRPHGSSRLQERAAVDTFINTEKPIAWNKLLANIGPNGKAAPGAAAGVVIASPSRTDPPYFFTWTRDAALVLTGIIESLGHNYNTTLQTVIQNYVASQAKLQQVSNPSGTFADGSGLGEAKFNVDLTAFTGEWGRPQRDGPPLRAIALIQYAKWLIANGYKSTAKSVVWPVVKNDLAYTAQYWNETGFDLWEEVPGSSFFTIASSHRALTEGAYLAAQLDTECRACTTVAPQVLCFQQAFWNSKGNYVVSNINGGEYRSGKDANSILASIHNFDPEAGCDNLTFQPCSERALANHKAYVDSFRNLYAINKGIAQGKAVAVGRYSEDVYYNGNPWYLANFAAAEQLYDAIYVWNKQGSITVTSVSLPFFRDLVSSVSTGTYSKSSSTFTNIVNAVKAYADGFIEVAAKYTPSNGALAEQYDRNTGKPDSAADLTWSYSAFLSAIDRRAGLVPPSWRASVAKSQLPSTCSRIEVAGTYVAATSTSFPSKQTPNPSAAPSPSPYPTACADASEVYVTFNERVSTAWGETIKVVGNVPALGNWDTSKAVTLSASGYKSNDPLWSITVPIKATGSAVQYKYIKVGTNGKITWESDPNRSITLQTASSAGKCAAQTVNDSWR

SEQ ID NO: 3 - protein, 604 aa, Humicola grisea
AAVDTFINTEKPIAWNKLLANIGPNGKAAPGAAAGVVIASPSRTDPPYFFTWTRDAALVLTGIIESLGHNYNTTLQTVIQNYVASQAKLQQVSNPSGTFADGSGLGEAKFNVDLTAFTGEWGRPQRDGPPLRAIALIQYAKWLIANGYKSTAKSVVWPVVKNDLAYTAQYWNETGFDLWEEVPGSSFFTIASSHRALTEGAYLAAQLDTECRACTTVAPQVLCFQQAFWNSKGNYVVSNINGGEYRSGKDANSILASIHNFDPEAGCDNLTFQPCSERALANHKAYVDSFRNLYAINKGIAQGKAVAVGRYSEDVYYNGNPWYLANFAAAEQLYDAIYVWNKQGSITVTSVSLPFFRDLVSSVSTGTYSKSSSTFTNIVNAVKAYADGFIEVAAKYTPSNGALAEQYDRNTGKPDSAADLTWSYSAFLSAIDRRAGLVPPSWRASVAKSQLPSTCSRIEVAGTYVAATSTSFPSKQTPNPSAAPSPSPYPTACADASEVYVTFNERVSTAWGETIKVVGNVPALGNWDTSKAVTLSASGYKSNDPLWSITVPIKATGSAVQYKYIKVGTNGKITWESDPNRSITLQTASSAGKCAAQTVNDSWR

SEQ ID NO: 4 - DNA, 1899 bp, Trichoderma reesei
atgcacgtcctgtcgactgcggtgctgctcggctccgttgccgttcaaaaggtcctgggaagaccaggatcaagcggtctgtccgacgtcaccaagaggtctgttgacgacttcatcagcaccgagacgcctattgcactgaacaatcttctttgcaatgttggtcctgatggatgccgtgcattcggcacatcagctggtgcggtgattgcatctcccagcacaattgacccggactactattacatgtggacgcgagatagcgctcttgtcttcaagaacctcatcgaccgcttcaccgaaacgtacgatgcgggcctgcagcgccgcatcgagcagtacattactgcccaggtcactctccagggcctctctaacccctcgggctccctcgcggacggctctggtctcggcgagcccaagtttgagttgaccctgaagcctttcaccggcaactggggtcgaccgcagcgggatggcccagctctgcgagccattgccttgattggatactcaaagtggctcatcaacaacaactatcagtcgactgtgtccaacgtcatctggcctattgtgcgcaacgacctcaactatgttgcccagtactggaaccaaaccggctttgacctctgggaagaagtcaatgggagctcattctttactgttgccaaccagcaccgagcacttgtcgagggcgccactcttgctgccactcttggccagtcgggaagcgcttattcatctgttgctccccaggttttgtgctttctccaacgattctgggtgtcgtctggtggatacgtcgactccaacatcaacaccaacgagggcaggactggcaaggatgtcaactccgtcctgacttccatccacaccttcgatcccaaccttggctgtgacgcaggcaccttccagccatgcagtgacaaagcgctctccaacctcaaggttgttgtcgactccttccgctccatctacggcgtgaacaagggcattcctgccggtgctgccgtcgccattggccggtatgcagaggatgtgtactacaacggcaacccttggtatcttgctacatttgctgctgccgagcagctgtacgatgccatctacgtctggaagaagacgggctccatcacggtgaccgccacctccctggccttcttccaggagcttgttcctggcgtgacggccgggacctactccagcagctcttcgacctttaccaacatcatcaacgccgtctcgacatacgccgatggcttcctcagcgaggctgccaagtacgtccccgccgacggttcgctggccgagcagtttgaccgcaacagcggcactccgctgtctgcgcttcacctgacgtggtcgtacgcctcgttcttgacagccacggcccgtcgggctggcatcgtgcccccctcgtgggccaacagcagcgctagcacgatcccctcgacgtgctccggcgcgtccgtggtcggatcctactcgcgtcccaccgccacgtcattccctccgtcgcagacgcccaagcctggcgtgccttccggtactccctacacgcccctgccctgcgcgaccccaacctccgtggccgtcaccttccacgagctcgtgtcgacacagtttggccagacggtcaaggtggcgggcaacgccgcggccctgggcaactggagcacgagcgccgccgtggctctggacgccgtcaactatgccgataaccaccccctgtggattgggacggtcaacctcgaggctggagacgtcgtggagtacaagtacatcaatgtgggccaagatggctccgtgacctgggagagtgatcccaaccacacttacacggttcctgcggtggcttgtgtgacgcaggttgtcaaggaggacacctggcagtcgtaa

SEQ ID NO: 5 - protein, 632 aa, Trichoderma reesei
MHVLSTAVLLGSVAVQKVLGRPGSSGLSDVTKRSVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 6 - protein, 599 aa, Trichoderma reesei
SVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 7 - protein, 453 aa, Trichoderma reesei
SVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTC

SEQ ID NO: 8 - protein, 604 aa, Rhizopus oryzae
MQLFNLPLKVSFFLVLSYFSLLVSAASIPSSASVQLDSYNYDGSTFSGKIYVKNIAYSKKVTVIYADGSDNWNNNGNTIAASYSAPISGSNYEYWTFSASINGIKEFYIKYEVSGKTYYDNNNSANYQVSTSKPTTTTATATTTTAPSTSTTTPPSRSEPATFPTGNSTISSWIKKQEGISRFAMLRNINPPGSATGFIAASLSTAGPDYYYAWTRDAALTSNVIVYEYNTTLSGNKTILNVLKDYVTFSVKTQSTSTVCNCLGEPKFNPDASGYTGAWGRPQNDGPAERATTFILFADSYLTQTKDASYVTGTLKPAIFKDLDYVVNVWSNGCFDLWEEVNGVHFYTLMVMRKGLLLGADFAKRNGDSTRASTYSSTASTIANKISSFWVSSNNWIQVSQSVTGGVSKKGLDVSTLLAANLGSVDDGFFTPGSEKILATAVAVEDSFASLYPINKNLPSYLGNSIGRYPEDTYNGNGNSQGNSWFLAVTGYAELYYRAIKEWIGNGGVTVSSISLPFFKKFDSSATSGKKYTVGTSDFNNLAQNIALAADRFLSTVQLHAHNNGSLAEEFDRTTGLSTGARDLTWSHASLITASYAKAGAPAA

SEQ ID NO: 9 - protein, 579 aa, Rhizopus oryzae
ASIPSSASVQLDSYNYDGSTFSGKIYVKNIAYSKKVTVIYADGSDNWNNNGNTIAASYSAPISGSNYEYWTFSASINGIKEFYIKYEVSGKTYYDNNNSANYQVSTSKPTTTTATATTTTAPSTSTTTPPSRSEPATFPTGNSTISSWIKKQEGISRFAMLRNINPPGSATGFIAASLSTAGPDYYYAWTRDAALTSNVIVYEYNTTLSGNKTILNVLKDYVTFSVKTQSTSTVCNCLGEPKFNPDASGYTGAWGRPQNDGPAERATTFILFADSYLTQTKDASYVTGTLKPAIFKDLDYVVNVWSNGCFDLWEEVNGVHFYTLMVMRKGLLLGADFAKRNGDSTRASTYSSTASTIANKISSFWVSSNNWIQVSQSVTGGVSKKGLDVSTLLAANLGSVDDGFFTPGSEKILATAVAVEDSFASLYPINKNLPSYLGNSIGRYPEDTYNGNGNSQGNSWFLAVTGYAELYYRAIKEWIGNGGVTVSSISLPFFKKFDSSATSGKKYTVGTSDFNNLAQNIALAADRFLSTVQLHAHNNGSLAEEFDRTTGLSTGARDLTWSHASLITASYAKAGAPAA

(19) United States
(12) Patent Application Publication
(10) Pub. No.: US 2012/0034659 A1
(43) Pub. Date: Feb. 9, 2012

(54) NEUTRAL pH SACCHARIFICATION AND FERMENTATION

(75) Inventors: Martien H. Bergsma, Zoetermeer (NL); Gopal K. Chotani, Cupertino, CA (US); William A. Cuevas, San Francisco, CA (US); Gang Duan, Shanghai (CN); Sung Ho Lee, North Liberty, IA (US); Ying Qian, Wuxi Jiangsu (CN); Vivek Sharma, North Liberty, IA (US); Jayarama K. Shetty, Pleasanton, CA (US); Bruce A. Strohm, Beloit, WI (US); Paula Johanna Maria Teunissen, Saratoga, CA (US); Hongxian Xu, Wuxi City (CN)

(73) Assignee: Danisco US Inc., Palo Alto, CA (US)

(21) Appl. No.: 13/204,561

(22) Filed: Aug. 5, 2011

Related U.S. Application Data
(60) Provisional application No. 61/371,639, filed on Aug. 6, 2010.

(51) Int. Cl. C12P 19/20; C12P 7/04; C12P 7/16; C12P 7/56; C12P 7/46; C12P 7/18; C12P 13/04; C12P 7/14; C12P 13/14

(52) U.S. Cl. 435/96; 435/162; 435/157; 435/160; 435/110; 435/145; 435/158; 435/106; 435/139

(57) ABSTRACT

Embodiments of the present disclosure relate to a process for producing downstream products, such as fermentable sugars and end products, from a starch substrate by saccharification and/or fermentation. The saccharification is effectively catalyzed by a glucoamylase at a pH in the range of 5.0 to 8.0. At a pH of 6.0 or above, the glucoamylase possesses at least 50% activity relative to its maximum activity. The saccharification and fermentation may be performed as a simultaneous saccharification and fermentation (SSF) process.

### CROSS-REFERENCE TO RELATED APPLICATIONS

This application claims the priority benefit of U.S. Provisional Appl. 61/371,639, filed Aug. 6, 2010, the contents of which are hereby incorporated by reference in its entirety.

### SEQUENCE LISTING

A Sequence Listing, comprising SEQ ID NOs: 1-9, is attached and incorporated herein by reference in its entirety.

### FIELD OF THE INVENTION

Glucoamylases capable of effectively hydrolyzing a starch substrate at a pH in the range of 5.0 to 8.0 are useful in simultaneous saccharification and fermentation (SSF) to product an end product.

### BACKGROUND

Industrial fermentations predominately use glucose as a feedstock for the production of a multitude of proteins, enzymes, alcohols, and other chemical end products. Typically, glucose is the product of starch processing, which is conventionally a two-step, enzymatic process that catalyzes the breakdown of starch, involving liquefaction and saccharification. During liquefaction, insoluble granular starch is slurried in water, gelatinized with heat, and hydrolyzed by a thermostable alpha-amylase. During saccharification, the soluble dextrins produced in liquefaction are further hydrolyzed by glucoamylases producing a high glucose syrup containing greater than 95% glucose.

Glucoamylases are exo-acting carbohydrases, capable of hydrolyzing both the linear and branched glucosidic linkages of starch (e.g., amylose and amylopectin). Commercially, glucoamylases are typically used in the acidic pH ranges (pH less than 5.0) to produce fermentable sugars from the enzyme liquefied starch substrate. The fermentable sugars, e.g., low molecular weight sugars, such as glucose, may then be converted to fructose by other enzymes (e.g., glucose isomerases); crystallized; or used in fermentations to produce numerous end products (e.g., alcohols, monosodium glutamate, succinic acid, vitamins, amino acids, 1,3-propanediol, and lactic acid).

A system that combines (1) saccharification and (2) fermentation is known as simultaneous saccharification and fermentation (SSF). SSF replaces the classical double-step fermentation, i.e., production of fermentable sugars first and then conducting the fermentation process for producing the end product. In SSF, an inoculum can be added along with the starch hydrolyzing enzymes to concurrently saccharify a starch substrate and convert the saccharification products (i.e., fermentable sugars) to the desired end product. The inoculum is typically a microorganism capable of producing the end product. The benefits of SSF include, but are not limited to, the following:
1) promote sustained microbial growth by providing a continuous feeding of glucose;
2) improving the carbon conversion efficiency by reducing the osmotic stress;
3) boosting fermentation capacity by accommodating higher dry solids; and
4) improving end product yield and facilitating downstream processing due to reduced production of reversion reaction products and non-fermentable sugars.
   SSF is particularly promising where a high concentration substrate is present in a low reactor volume. See e.g., John et al., Biotechnol. Adv. 27: 145-152 (2009).

There is still need to optimize SSF by choosing conditions, e.g., temperature, pH, enzymes, etc., that are most suitable for both saccharification and fermentation. For example, the pH of the yeast fermentation matches the saccharifying glucoamylase enzyme activity during the production of fuel alcohol using grain as a feedstock. The need is acute particularly for fermentations that are optimally performed above pH 6.0. Most commercial saccharification enzymes, e.g., Aspergillus niger glucoamylase (AnGA), only display significant saccharifying enzyme activity in the pH range of 4.2 to 5.5. The glucoamylases display significantly lowered activity at the fermentation pH above 6.0. Additional steps, such as pre-treatment or pre-saccharification for producing fermentation feed stocks, have been used when the optimal conditions for the fermentation and saccharification are not congruent. As disclosed in WO 2003/066816, for example, the slurry is subject to pasteurization at 65° C. for 14 hours before the SSF is performed to produce 1,3-propanediol at 34° C. Similarly, the lactic acid-producing microorganism may be subject to forty to fifty serial transfers at an acidic pH before being applied in the SSF to produce lactic acid. See WO 2003/095659.

### SUMMARY OF THE INVENTION

Glucoamylases such as Humicola grisea glucoamylase (HgGA), Trichoderma reesei glucoamylase (TrGA), and Rhizopus oryzae/niveus. glucoamylase (RhGA) display different pH profiles from other known glucoamylases, such as glucoamylases (GAs) from Aspergillus niger (AnGA) and Talaromyces emersonii (TeGA). At a pH of 6.0 or above, both HgGA and TrGA retain at least 50% of the activity relative to the maximum activity at pH 4.25 or pH 3.75, respectively. Both HgGA and TrGA are capable of saccharifying a starch substrate effectively at a pH in the range of 5.0 to 8.0. This property enables HgGA and TrGA to be used in simultaneous saccharification and fermentation (SSF) to produce end products from a starch substrate.

The embodiment contemplated herein provides a method of processing starch to produce fermentable sugars at pH 5.0 to 8.0. The fermentable sugars are produced by saccharifying a starch substrate in the presence of a glucoamylase, which possesses at least 50% activity at pH 6.0 or above relative to its maximum activity. Saccharifying may be carried out at a pH in a range of 6.0 to 7.5, or optionally 7.0 to 7.5. Saccharifying is performed at a temperature in a range of about 30° C. to about 60° C., or optionally about 30° C. to about 40° C.

In one aspect, the starch substrate is from corn, wheat, rye, barley, sorghum, cassava, tapioca, potato and any combination thereof. In another aspect, the starch substrate is granular starch or liquefied starch. In a further aspect, the starch substrate is about 15% to 50%, about 15% to 30%, or about 15% to 25% dry solid (DS).

In one embodiment, the method further comprises fermenting the fermentable sugars to an end product at the same pH saccharifying is performed. The end product may be selected from the group consisting of methanol, ethanol, butanol, monosodium glutamate, succinic acid, 1,3-propanediol, vitamins, amino acids, and lactic acid. Optionally, the end product is ethanol, 1,3-propanediol, or succinic acid. In another embodiment, saccharifying and fermenting are carried out as a simultaneous saccharification and fermentation (SSF) process, which is typically carried out at pH 6.5 to 7.5.

In one aspect, the glucoamylase is selected from the group consisting of Humicola grisea glucoamylase (HgGA) comprising SEQ ID NO: 3, Trichoderma reesei glucoamylase (TrGA) comprising SEQ ID NO: 6, Rhizopus p. glucoamylase (RhGA) comprising SEQ ID NO: 9, and a variant thereof. The variant has at least 99% sequence identity to the parent glucoamylase. Optionally, the variant has one amino acid modifications compared to the parent glucoamylase. In another aspect, the HgGA is SEQ ID NO: 3, and is optionally produced from a Trichoderma reesei host cell. In a further aspect, the TrGA is SEQ ID No: 6. In yet another aspect, the RhGA is SEQ ID NO: 9.

In one embodiment, the glucoamylase is added at a range of about 0.1 to about 2.0, about 0.2 to about 1.0, or 0.5 to 1.0 GAU per gram of dry substance starch. In another embodiment, saccharifying further comprises adding an alpha-amylase. The alpha-amylase is from a Bacillus species, or a variant thereof. The alpha-amylase is a Bacillus subtilis alpha-amylase (AmyE), a Bacillus amyloliquefaciens alpha-amylase, a Bacillus licheniformis alpha-amylase, a Bacillus stearothermophilus alpha-amylase, or a variant thereof.

In another embodiment, the invention provides for methods of processing starch comprising saccharifying a starch substrate to fermentable sugars at pH 5.0 to 8.0 in the presence of glucoamylase and at least one other enzyme, wherein the glucoamylase possesses at least 50% activity at pH 6.0 or above relative to its maximum activity, wherein the glucoamylase is selected from the group consisting of Humicola grisea glucoamylase (HgGA) comprising SEQ ID NO: 3, Trichoderma reesei glucoamylase (TrGA) comprising SEQ ID NO: 6, Rhizopus sp. glucoamylase (RhGA) comprising SEQ ID NO: 9, and a variant thereof, and wherein the variant has at least 99% sequence identity to a parent glucoamylase, and wherein the other enzyme is selected from the group consisting of proteases, pullulanases, isoamylases, cellulases, hemicellulases, xylanases, cyclodextrin glycotransferases, lipases, phytases, laccases, oxidases, esterases, cutinases, xylanases, and alpha-glucosidases.

In another embodiment, the invention provides for methods of processing starch comprising saccharifying a starch substrate to fermentable sugars at pH 5.0 to 8.0 in the presence of glucoamylase and at least one other non-starch polysaccharide hydrolyzing enzymes, wherein the glucoamylase possesses at least 50% activity at pH 6.0 or above relative to its maximum activity, wherein the glucoamylase is selected from the group consisting of Humicola grisea glucoamylase (HgGA) comprising SEQ ID NO: 3, Trichoderma reesei glucoamylase (TrGA) comprising SEQ ID NO: 6, Rhizopus sp. glucoamylase (RhGA) comprising SEQ ID NO: 9, and a variant thereof, and wherein the variant has at least 99% sequence identity to a parent glucoamylase, and wherein the non-starch polysaccharide hydrolyzing enzymes is selected from the group consisting of cellulases, hemicellulases and pectinases.

### BRIEF DESCRIPTION OF THE DRAWINGS

The accompanying drawings are incorporated into the specification and provide non-limiting illustrations of various embodiments. In the drawings:

FIG. 1 depicts the pH profiles of HgGA, TrGA, AnGA, and TeGA, at 32° C. The pH profiles are presented as the percentage of the maximum activity under the saccharification conditions described in Example 1.

FIG. 2 depicts the presence of higher sugars after 48-hour saccharification reactions catalyzed by HgGA, TrGA, and AnGA. The saccharification reactions are described in Example 4.

FIG. 3 depicts scanning electron micrographs of corn, wheat, and cassava starch treated with HgGA and an alpha-amylase at pH 6.4. Starch samples are hydrolyzed by HgGA and an alpha-amylase under the conditions as described in Example 7.

### DETAILED DESCRIPTION

The present disclosure relates to a glucoamylase capable of effectively saccharifying a starch substrate at a neutral pH, for example, between pH 5.0 and 8.0. At a pH of 6.0 or above, the glucoamylase retains at least about 50% activity relative to the maximum activity. The glucoamylases having the unusually properties may include, for example, HgGA, TrGA, and RhGA. Also disclosed includes a method of using the glucoamylase to perform simultaneous saccharification and fermentation (SSF), at a neutral pH, to produce an end product, for example, 1,3-propanediol, succinic acid, lysine, monosodium glutamate, and lactic acid.

In some aspects, the embodiments of the present disclosure rely on routine techniques and methods used in the field of genetic engineering and molecular biology. The following resources include descriptions of general methodology useful in accordance with the invention: Sambrook et al., MOLECULAR CLONING: A LABORATORY MANUAL (2nd Ed., 1989); Kreigler, GENE TRANSFER AND EXPRESSION; A LABORATORY MANUAL (1990) and Ausubel et al., Eds. CURRENT PROTOCOLS IN MOLECULAR BIOLOGY (1994). Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with a general dictionary of many of the terms used in this invention. Although any methods and materials similar or equivalent to those described herein can be used in the practice or testing of the present invention, the representative methods and materials are described. Numeric ranges are inclusive of the numbers defining the range. The headings provided herein are not limitations of the various aspects or embodiments, which can be had by reference to the specification as a whole.

### 1. Definitions and Abbreviations

In accordance with this detailed description, the following abbreviations and definitions apply. It should be noted that as used herein, the singular forms “a,” “an,” and “the” include plural referents unless the context clearly dictates otherwise. Thus, for example, reference to “an enzyme” includes a plurality of such enzymes.

### 1.1. Definitions

As used herein, “amino acid sequence” is synonymous with the term “polypeptide” and/or the term “protein.” In some instances, the term “amino acid sequence” is synonymous with the term “peptide”; in some instances, the term “amino acid sequence” is synonymous with the term “enzyme.”

As used herein, “nucleotide sequence” or “nucleic acid sequence” refers to a sequence of genomic, synthetic, or recombinant origin and may be double-stranded or single-stranded, whether representing the sense or anti-sense strand. As used herein, the term “nucleic acid” may refer to genomic DNA, cDNA, synthetic DNA, or RNA. The residues of a nucleic acid may contain any of the chemically modifications commonly known and used in the art.

“Isolated” means that the material is at least substantially free from at least one other component that the material is naturally associated and found in nature.

“Purified” means that the material is in a relatively pure state, e.g., at least about 90% pure, at least about 95% pure, at least about 98% pure, or at least about 99% pure.

“Oligosaccharide” means a carbohydrate molecule composed of 3-20 monosaccharides.

As used herein, “transformed cell” includes cells that have been transformed by use of recombinant DNA techniques. Transformation typically occurs by insertion of one or more nucleotide sequences into a cell. The inserted nucleotide sequence may be a heterologous nucleotide sequence, i.e., is a sequence that may not be natural to the cell that is to be transformed, such as a fusion protein.

As used herein, “starch” refers to any material comprised of the complex polysaccharide carbohydrates of plants, comprised of amylose and amylopectin with the formula (C₆H₁₀O₅)ₓ, wherein “X” can be any number. In particular, the term refers to any plant-based material including but not limited to grains, grasses, tubers and roots and more specifically wheat, barley, corn, rye, rice, sorghum, brans, cassava, millet, potato, sweet potato, and tapioca.

As used herein, “granular starch” refers to uncooked (raw) starch, which has not been subject to gelatinization.

As used herein, “starch gelatinization” means solubilization of a starch molecule to form a viscous suspension.

As used herein, “gelatinization temperature” refers to the lowest temperature at which gelatinization of a starch substrate occurs. The exact temperature depends upon the specific starch substrate and further may depend on the particular variety and the growth conditions of plant species from which the starch is obtained.

“DE” or “dextrose equivalent” is an industry standard for measuring the concentration of total reducing sugars, calculated as the percentage of the total solids that have been converted to reducing sugars. The granular starch that has not been hydrolyzed has a DE that is about zero (0), and D-glucose has a DE of about 100.

As used herein, “starch substrate” refers to granular starch or liquefied starch using refined starch, whole ground grains, or fractionated grains.

As used herein, “liquefied starch” refers to starch that has gone through solubilization process, for example, the conventional starch liquefaction process.

As used herein, “glucose syrup” refers to an aqueous composition containing glucose solids. Glucose syrup will have a DE of at least about 20. In some embodiments, glucose syrup may contain no more than about 21% water while at least about 25% reducing sugar calculated as dextrose. In one embodiment, glucose syrup may include at least about 90% D-glucose, and in another embodiment, glucose syrup may include at least about 95% D-glucose. In some embodiments, the terms glucose and glucose syrup are used interchangeably.

“Degree of polymerization (DP)” refers to the number (n) of anhydroglucopyranose units in a given saccharide. Examples of DP1 are the monosaccharides, such as glucose and fructose. Examples of DP2 are the disaccharides, such as maltose and sucrose. A DP4+ (>DP4) denotes polymers with a degree of polymerization of greater than four.

As used herein, “fermentable sugars” refer to saccharides that are capable of being metabolized under fermentation conditions. These sugars typically refer to glucose, maltose, and maltotriose (DP1, DP2 and DP3).

As used herein, “total sugar content” refers to the total sugar content present in a starch composition.

As used herein, “ds” refers to dissolved solids in a solution. The term “dry solids content (DS)” refers to the total solids of a slurry in % on a dry weight basis. The term “slurry” refers to an aqueous mixture containing insoluble solids.

As used herein, “starch-liquefying enzyme” refers to an enzyme that catalyzes the hydrolysis or breakdown of granular starch. Exemplary starch liquefying enzymes include alpha-amylases (EC 3.2.1.1).

“Amylase” means an enzyme that is, among other things, capable of catalyzing the degradation of starch. For example, β-Amylases, α-glucosidases (EC 3.2.1.20; α-D-glucoside glucohydrolase), glucoamylase (EC 3.2.1.3; α-D-(1→4)-glucan glucohydrolase), and product-specific amylases can produce malto-oligosaccharides of a specific length from starch.

“Alpha-amylases (EC 3.2.1.1)” refer to endo-acting enzymes that cleave α-D-(1→4) O-glycosidic linkages within the starch molecule in a random fashion. In contrast, the exo-acting amylolytic enzymes, such as beta-amylases (EC 3.2.1.2; α-D-(1→4)-glucan maltohydrolase) and some product-specific amylases like maltogenic alpha-amylase (EC 3.2.1.133) cleave the starch molecule from the non-reducing end of the substrate. These enzymes have also been described as those effecting the exo- or endohydrolysis of 1,4-α-D-glucosidic linkages in polysaccharides containing 1,4-α-linked D-glucose units. Another term used to describe these enzymes is glycogenase. Exemplary enzymes include alpha-1,4-glucan 4-glucanohydrolase.

As used herein, “glucoamylases” refer to the amyloglucosidase class of enzymes (EC 3.2.1.3, glucoamylase, α-1,4-D-glucan glucohydrolase). These are exo-acting enzymes that release glucosyl residues from the non-reducing ends of amylose and/or amylopectin molecules. The enzymes are also capably of hydrolyzing α-1, 6 and α-1,3 linkages, however, at much slower rates than the hydrolysis of α-1,4 linkages.

As used herein, the term “non-starch polysaccharide hydrolyzing enzymes” are enzymes capable of hydrolyzing complex carbohydrate polymers such as cellulose, hemicellulose, and pectin. For example, cellulases (endo and exo-glucanases, beta glucosidase) hemicellulases (xylanases) and pectinases are non-starch polysaccharide hydrolyzing enzymes.

As used herein, “maximum activity” refers to the enzyme activity measured under the most favorable conditions, for example, at an optimum pH. As used herein, “optimum pH” refers to a pH value, under which the enzyme displays the highest activity with other conditions being equal.

The phrase “mature form” of a protein or polypeptide refers to the final functional form of the protein or polypeptide. A mature form of a glucoamylase may lack a signal peptide and/or initiator methionine, for example. A mature form of a glucoamylase may be produced from its native host, for example, by endogenous expression. Alternatively, a mature form of a glucoamylase may be produced from a non-native host, for example, by exogenous expression. An exogenously expressed glucoamylase, while maintaining the glucoamylase activity, may have a varied glycosylation pattern compared to the endogenous expressed counterpart.

The term “parent” or “parent sequence” refers to a sequence that is native or naturally occurring in a host cell.

As used herein, the terms “variant” is used in reference to glucoamylases that have some degree of amino acid sequence identity to a parent glucoamylase sequence. A variant is similar to a parent sequence, but has at least one substitution, deletion or insertion in their amino acid sequence that makes them different in sequence from a parent glucoamylase. In some cases, variants have been manipulated and/or engineered to include at least one substitution, deletion, or insertion in their amino acid sequence that makes them different in sequence from a parent. Additionally, a glucoamylase variant may retain the functional characteristics of the parent glucoamylase, e.g., maintaining a glucoamylase activity that is at least about 50%, about 60%, about 70%, about 80%, or about 90% of that of the parent glucoamylase.

As used herein, “hydrolysis of starch” refers to the cleavage of glucosidic bonds with the addition of water molecules.

As used herein, “no-cook” refers to a process of converting a granular starch substrate, for example, raw starch, to fermentable sugars without the conventional high-temperature starch liquefaction process.

As used herein, “end product” or “desired end product” refers to a molecule or compound to which a starch substrate is converted into, by an enzyme and/or a microorganism.

As used herein, “contacting” or “admixing” refers to the placing of the respective enzyme(s) in sufficiently close proximity to the respective substrate to enable the enzyme(s) to convert the substrate to the end product. Those skilled in the art will recognize that mixing solutions of the enzyme with the respective substrates can affect contacting or admixing.

### 1.2. Abbreviations

The following abbreviations apply unless indicated otherwise:

AkAA Aspergillus kawachii alpha-amylase

AmyE Bacillus subtilis alpha-amylase

AmyL Bacillus licheniformis alpha-amylase

AmyR SPEZYME® XTRA amylase

AmyS Geobacillus stearothermophilus alpha-amylase

AnGA Aspergillus niger glucoamylase

BAA bacterial alpha-amylase

cDNA complementary DNA

DE Dextrose Equivalent

DI distilled, deionized

DNA deoxyribonucleic acid

DP3 degree of polymerization with three subunits

DPn degree of polymerization with n subunits

DS or ds dry solid

dss dry solid starch

EC enzyme commission for enzyme classification

g gram

gpm gallon per minute

GAU glucoamylase units

HGA Humicola grisea glucoamylase

HgGA Humicola grisea glucoamylase

HPLC high pressure liquid chromatography

kg kilogram

MOPS 3-(N-morpholino)propanesulfonic acid

MT metric ton

MW molecular weight

NCBI National Center for Biotechnology Information

nm nanometer

OD optical density

PCR polymerase chain reaction

PEG polyethylene glycol

pI isoelectric point

ppm parts per million

RhGA Rhizopus sp. glucoamylase

RNA ribonucleic acid

RO reverse osmosis

rpm revolutions per minute

slpm standard liters per minute

SSF simultaneous saccharification and fermentation

TeGA Talaromyces emersonii glucoamylase

TrGA Trichoderma reesei glucoamylase

w/v weight/volume

w/w weight/weight

wt wild-type

μL microliter

### 2. Enzymes in Starch Processing

2.1. Glucoamylase

2.1.1. Structure and Function

Glucoamylases are produced by numerous strains of bacteria, fungi, yeast and plants. Many fungal glucoamylases are fungal enzymes that are extracellularly produced, for example from strains of Aspergillus (Svensson et al., Carlsberg Res. Commun. 48: 529-544 (1983); Boel et al., EMBO J. 3: 1097-1102 (1984); Hayashida et al., Agric. Biol. Chem. 53: 923-929 (1989); U.S. Pat. No. 5,024,941; U.S. Pat. No. 4,794,175 and WO 88/09795); Talaromyces (U.S. Pat. No. 4,247,637; U.S. Pat. No. 6,255,084; and U.S. Pat. No. 6,620,924); Rhizopus (Ashikari et al., Agric. Biol. Chem. 50: 957-964 (1986); Ashikari et al., App. Microbio. Biotech. 32: 129-133 (1989) and U.S. Pat. No. 4,863,864); Humicola (WO 05/052148 and U.S. Pat. No. 4,618,579); and Mucor (Houghton-Larsen et al., Appl. Microbiol. Biotechnol. 62: 210-217 (2003)). Many of the genes that code for these enzymes have been cloned and expressed in yeast, fungal and/or bacterial cells.

Commercially, glucoamylases are very important enzymes and have been used in a wide variety of applications that require the hydrolysis of starch (e.g., for producing glucose and other monosaccharides from starch). Glucoamylases are used to produce high fructose corn sweeteners, which comprise over 50% of the sweetener market in the United States. In general, glucoamylases may be, and commonly are, used with alpha-amylases in starch hydrolyzing processes to hydrolyze starch to dextrins and then glucose. The glucose may then be converted to fructose by other enzymes (e.g., glucose isomerases); crystallized; or used in fermentations to produce numerous end products (e.g., ethanol, citric acid, succinic acid, ascorbic acid intermediates, glutamic acid, glycerol, 1,3-propanediol and lactic acid).

Glucoamylases consist of as many as three distinct structural domains, a catalytic domain of approximately 450 residues that is structurally conserved in all glucoamylases, generally followed by a linker region consisting of between 30 and 80 residues that are connected to a starch binding domain of approximately 100 residues. The structure of the Trichoderma reesei glucoamylase (TrGA) with all three regions intact was determined to 1.8 Angstrom resolution. See WO 2009/048488 and WO 2009/048487. Using the determined coordinates, the structure was aligned with the coordinates of the catalytic domain of the glucoamylase from Aspergillus awamori strain X100 that was determined previously (Aleshin, A. E., Hoffman, C., Firsov, L. M., and Honzatko, R. B. Refined crystal structures of glucoamylase from Aspergillus awamori var. X100. J. Mol. Biol. 238: 575-591 (1994)). See id. The structure of the catalytic domains of these two glucoamylases overlap very closely, and it is possible to identify equivalent residues based on this structural superposition. See id. It is further believed that all glucoamylases share the basic structure. See id.

Given the well-known structure and function relationship of glucoamylases, glucoamylase variants having altered properties have been successfully created and characterized. The variants may display improved properties as compared to the parent glucoamylases. The improved properties may include and are not limited to increased thermostability and increased specific activity. For example, methods for making and characterizing TrGA variants with altered properties have been described in WO 2009/067218.

2.1.2. Glucoamylases Having the Desired pH Profile

The embodiments of the present disclosure utilize a glucoamylase capable of effectively saccharifying a starch substrate at a neutral pH, for example, between pH 5.0 and 8.0, 5.5 and 7.5, 6.0 and 7.5, 6.5 and 7.5, or 7.0 and 7.5. At a pH of 6.0 or above, the glucoamylase retains at least about 50%, about 51%, about 52%, about 53%, about 54%, or about 55% of the activity relative to the maximum activity. The glucoamylases having the desired pH profile include, but are not limited to, Humicola grisea glucoamylase (HgGA), Trichoderma reesei glucoamylase (TrGA), and Rhizopus sp. glucoamylase (RhGA).

HgGA may be the glucoamylase comprising the amino acid sequence of SEQ ID NO: 3, which is described in detail in U.S. Pat. Nos. 4,618,579 and 7,262,041. This HgGA is also described as a granular starch hydrolyzing enzyme (GSHE), because it is capable of hydrolyzing starch in granular form. The genomic sequence coding the HgGA from Humicola grisea var. thermoidea is presented as SEQ ID NO: 1, which contains three putative introns (positions 233-307, 752-817, and 950-1006). The native HgGA from Humicola grisea var. thermoidea has the amino acid sequence of SEQ ID NO: 2, which includes a signal peptide containing 30 amino acid residues (positions 1 to 30 of SEQ ID NO: 2). Cleavage of the signal peptide results in the mature HgGA having the amino acid sequence of SEQ ID NO: 3. The embodiments of the present disclosure also include a HgGA produced from a Trichoderma host cell, e.g., a Trichoderma reesei cell. See U.S. Pat. No. 7,262,041.

TrGA may be the glucoamylase from Trichoderma reesei QM6a (ATCC, Accession No. 13631). This TrGA comprising the amino acid sequence of SEQ ID NO: 6, which is described in U.S. Pat. No. 7,413,879, for example. The cDNA sequence coding the TrGA from Trichoderma reesei QM6a is presented as SEQ ID NO: 4. The native TrGA has the amino acid sequence of SEQ ID NO: 5, which includes a signal peptide containing 33 amino acid residues (positions 1 to 33 of SEQ ID NO: 4). See id. Cleavage of the signal peptide results in the mature TrGA having the amino acid sequence of SEQ ID NO: 6. See id. The catalytic domain of TrGA is presented as SEQ ID NO: 7. See id. The embodiments of the present disclosure also include an endogenously expressed TrGA. See id.

RhGA may be the glucoamylase from Rhizopus niveus or Rhizopus oryzae. See U.S. Pat. Nos. 4,514,496 and 4,092,434. The native RhGA from R. oryzae has the amino acid sequence of SEQ ID NO: 8, which includes a signal peptide containing 25 amino acid residues (positions 1 to 25 of SEQ ID NO:8). Cleavage of the signal peptide results in the mature RhGA having the amino acid sequence of SEQ ID NO: 9. A typical RhGA may be the glucoamylase having trade names CU.CONC (Shin Nihon Chemicals, Japan) or M1 (Biocon India, Bangalore, India).

The glucoamylase of the embodiment of the present disclosure may also be a variant of HgGA, TrGA, or RhGA. The variant has at least 99% sequence identity to the parent glucoamylase. In some embodiments, the variant has at least 98%, at least 97%, at least 96%, at least 95%, at least 94%, at least 93%, at least 92%, at least 91%, or at least 90% sequence identity to the parent glucoamylase. Optionally, the variant has one, two, three, four, five, or six amino acids modification compared to the mature form of the parent glucoamylase. Optionally, the variant has more than six amino acids (e.g., 7, 8, 9, 10, 11, 12, 13, 14, 15, 20, 25, 30, 35, 40, 45, 50, 55, or 60) modification compared to the mature form of the parent glucoamylase. The variant possesses the desired pH profile and capability of saccharifying a starch substrate at a pH in the range of 5.0 to 8.0. In some embodiments, the variants may possess other improved properties, such as improved thermostability and improved specificity.

2.1.3. Production of Glucoamylase

Glucoamylases suitable for the embodiments of the present disclosure may be produce with recombinant DNA technology in various host cells.

In some embodiments, the host cells are selected from bacterial, fungal, plant and yeast cells. The term host cell includes both the cells, progeny of the cells and protoplasts created from the cells that are used to produce a variant glucoamylase according to the disclosure. In some embodiments, the host cells are fungal cells and typically filamentous fungal host cells. The term “filamentous fungi” refers to all filamentous forms of the subdivision Eumycotina (See, Alexopoulos, C. J. (1962), INTRODUCTORY MYCOLOGY, Wiley, New York). These fungi are characterized by a vegetative mycelium with a cell wall composed of chitin, cellulose, and other complex polysaccharides. The filamentous fungi of the present disclosure are morphologically, physiologically, and genetically distinct from yeasts. Vegetative growth by filamentous fungi is by hyphal elongation and carbon catabolism is obligatory aerobic. In the embodiments of the present disclosure, the filamentous fungal parent cell may be a cell of a species of, but not limited to, Trichoderma, (e.g., Trichoderma reesei, the asexual morph of Hypocrea jecorina, previously classified as T. longibrachiatum, Trichoderma viride, Trichoderma koningii, Trichoderma harzianum) (Sheir-Neirs et al., (1984) Appl. Microbiol. Biotechnol 20:46-53; ATCC No. 56765 and ATCC No. 26921); Penicillium sp., Humicola sp. (e.g., H. insolens, H. lanuginosa and H. grisea); Chrysosporium sp. (e.g., C. lucknowense), Gliocladium sp., Aspergillus sp. (e.g., A. oryzae, A. niger, A. sojae, A. japonicus, A. nidulans, and A. awamori) (Ward et al., (1993) Appl. Microbiol. Biotechnol. 39:738-743 and Goedegebuur et al., (2002) Genet. 41:89-98), Fusarium sp., (e.g., F. roseum, F. graminum F. cerealis, F. oxysporuim and F. venenatum), Neurospora sp., (N. crassa), Hypocrea sp., Mucor sp., (M. miehei), Rhizopus sp. and Emericella sp. (see also, Innis et al., (1985) Sci. 228:21-26). The term “Trichoderma” or “Trichoderma sp.” or “Trichoderma spp.” refers to any fungal genus previously or currently classified as Trichoderma. In other embodiments, the host cell will be a genetically engineered host cell wherein native genes have been inactivated, for example by deletion in fungal cells. Where it is desired to obtain a fungal host cell having one or more inactivated genes known methods may be used (e.g. methods disclosed in U.S. Pat. Nos. 5,246,853 and 5,475,101, and WO 92/06209). Gene inactivation may be accomplished by complete or partial deletion, by insertional inactivation or by any other means that renders a gene nonfunctional for its intended purpose (such that the gene is prevented from expression of a functional protein). In some embodiments, when the host cell is a Trichoderma cell and particularly a T. reesei host cell, the cbh1, cbh2, egl1 and egl2 genes will be inactivated and/or typically deleted. Typically, Trichoderma reesei host cells having quad-deleted proteins are set forth and described in U.S. Pat. No. 5,847,276 and WO 05/001036. In other embodiments, the host cell is a protease deficient or protease minus strain.

To produce the glucoamylase of the embodiments of the present disclosure with the recombinant DNA technology, a DNA construct comprising nucleic acid encoding the amino acid sequence of the designated glucoamylase can be constructed and transferred into, for example, a Trichoderma reesei host cell. The vector may be any vector which when introduced into a Trichoderma reesei host cell can be integrated into the host cell genome and can be replicated. Reference is made to the Fungal Genetics Stock Center Catalogue of Strains (FGSC, <www.fgsc.net>) for a list of vectors. Additional examples of suitable expression and/or integration vectors are provided in Sambrook et al., (1989) supra, and Ausubel (1987) supra, and van den Hondel et al. (1991) in Bennett and Lasure (Eds.) MORE GENE MANIPULATIONS IN FUNGI, Academic Press pp. 396 428 and U.S. Pat. No. 5,874,276. The nucleic acid encoding the glucoamylase can be operably linked to a suitable promoter, which shows transcriptional activity in Trichoderma reesei host cell. The promoter may be derived from genes encoding proteins either homologous or heterologous to the host cell. Suitable non-limiting examples of promoters include cbh1, cbh2, egl1, egl2. In one embodiment, the promoter may be a native T. reesei promoter. Typically, the promoter can be T. reesei cbh1, which is an inducible promoter and has been deposited in GenBank under Accession No. D86235. An “inducible promoter” may refer to a promoter that is active under environmental or developmental regulation. In another embodiment, the promoter can be one that is heterologous to T. reesei host cell. Other examples of useful promoters include promoters from A. awamori and A. niger glucoamylase genes (see, e.g., Nunberg et al., (1984) Mol. Cell Biol. 4:2306-2315 and Boel et al., (1984) EMBO J. 3:1581-1585). Also, the promoters of the T. reesei xln1 gene and the cellobiohydrolase 1 gene may be useful (EPA 13f280A1).

In some embodiments, the glucoamylase coding sequence can be operably linked to a signal sequence. The signal sequence may be the native signal peptide of the glucoamylase (residues 1-20 of SEQ ID NO: 2 for HgGA, or residues 1-33 of SEQ ID NO: 5 for TrGA, for example). Alternatively, the signal sequence may have at least 90% or at least 95% sequence identity to the native signal sequence. In additional embodiments, a signal sequence and a promoter sequence comprising a DNA construct or vector to be introduced into the T. reesei host cell are derived from the same source. For example, in some embodiments, the signal sequence can be the cdh1 signal sequence that is operably linked to a cdh1 promoter.

In some embodiments, the expression vector may also include a termination sequence. In one embodiment, the termination sequence and the promoter sequence can be derived from the same source. In another embodiment, the termination sequence can be homologous to the host cell. A particularly suitable terminator sequence can be cbh1 derived from T. reesei. Other exemplary fungal terminators include the terminator from A. niger or A. awamori glucoamylase gene.

In some embodiments, an expression vector may include a selectable marker. Examples of representative selectable markers include ones that confer antimicrobial resistance (e.g., hygromycin and phleomycin). Nutritional selective markers also find use in the present invention including those markers known in the art as amdS, argB, and pyr4. Markers useful in vector systems for transformation of Trichoderma are known in the art (see, e.g., Finkelstein, chapter 6 in BIOTECHNOLOGY OF FILAMENTOUS FUNGI, Finkelstein et al. Eds. Butterworth-Heinemann, Boston, Mass. (1992), Chap. 6; and Kinghorn et al. (1992) APPLIED MOLECULAR GENETICS OF FILAMENTOUS FUNGI, Blackie Academic and Professional, Chapman and Hall, London). In a representative embodiment, the selective marker may be the amdS gene, which encodes the enzyme acetamidase, allowing transformed cells to grow on acetamide as a nitrogen source. The use of A. nidulans amdS gene as a selective marker is described for example in Kelley et al., (1985) EMBO J. 4:475-479 and Penttila et al., (1987) Gene 61:155-164.

An expression vector comprising a DNA construct with a polynucleotide encoding the glucoamylase may be any vector which is capable of replicating autonomously in a given fungal host organism or of integrating into the DNA of the host. In some embodiments, the expression vector can be a plasmid. In typical embodiments, two types of expression vectors for obtaining expression of genes are contemplated.

The first expression vector may comprise DNA sequences in which the promoter, glucoamylase-coding region, and terminator all originate from the gene to be expressed. In some embodiments, gene truncation can be obtained by deleting undesired DNA sequences (e.g., DNA encoding unwanted domains) to leave the domain to be expressed under control of its own transcriptional and translational regulatory sequences.

The second type of expression vector may be preassembled and contains sequences needed for high-level transcription and a selectable marker. In some embodiments, the coding region for the glucoamylase gene or part thereof can be inserted into this general-purpose expression vector such that it is under the transcriptional control of the expression construct promoter and terminator sequences. In some embodiments, genes or part thereof may be inserted downstream of a strong promoter, such as the strong cbh1 promoter.

Methods used to ligate the DNA construct comprising a polynucleotide encoding the glucoamylase, a promoter, a terminator and other sequences and to insert them into a suitable vector are well known in the art. Linking can be generally accomplished by ligation at convenient restriction sites. If such sites do not exist, the synthetic oligonucleotide linkers are used in accordance with conventional practice. (see, Sambrook (1989) supra, and Bennett and Lasure, MORE GENE MANIPULATIONS IN FUNGI, Academic Press, San Diego (1991) pp 70-76.). Additionally, vectors can be constructed using known recombination techniques (e.g., Invitrogen Life Technologies, Gateway Technology).

Introduction of a DNA construct or vector into a host cell includes techniques such as transformation; electroporation; nuclear microinjection; transduction; transfection, (e.g., lipofection mediated and DEAE-Dextrin mediated transfection); incubation with calcium phosphate DNA precipitate; high velocity bombardment with DNA-coated microprojectiles; and protoplast fusion. General transformation techniques are known in the art (see, e.g., Ausubel et al., (1987), supra, chapter 9; and Sambrook (1989) supra, and Campbell et al., (1989) Curr. Genet. 16:53-56). The expression of heterologous protein in Trichoderma is described in U.S. Pat. Nos. 6,022,725; 6,268,328; Harkki et al. (1991); Enzyme Microb. Technol. 13:227-233; Harkki et al., (1989) Bio Technol. 7:596-603; EP 244,234; EP 215,594; and Nevalainen et al., “The Molecular Biology of Trichoderma and its Application to the Expression of Both Homologous and Heterologous Genes,” in MOLECULAR INDUSTRIAL MYCOLOGY, Eds. Leong and Berka, Marcel Dekker Inc., NY (1992) pp. 129-148).

In some embodiments, genetically stable transformants can be constructed with vector systems whereby the nucleic acid encoding glucoamylase is stably integrated into a host strain chromosome. Transformants are then purified by known techniques.

In one non-limiting example, stable transformants including an amdS marker are distinguished from unstable transformants by their faster growth rate and the formation of circular colonies with a smooth, rather than ragged outline on solid culture medium containing acetamide. Additionally, in some cases a further test of stability can be conducted by growing the transformants on solid non-selective medium (i.e., medium that lacks acetamide), harvesting spores from this culture medium and determining the percentage of these spores which subsequently germinate and grow on selective medium containing acetamide. Alternatively, other methods known in the art may be used to select transformants.

Uptake of DNA into the host Trichoderma sp. strain is dependent upon the calcium ion concentration. Generally, between about 10 mM CaCl₂ and 50 mM CaCl₂ may be used in an uptake solution. Besides the need for the calcium ion in the uptake solution, other compounds generally included are a buffering system such as TE buffer (10 mM Tris, pH 7.4; 1 mM EDTA) or 10 mM MOPS, pH 6.0 buffer (morpholinepropanesulfonic acid) and polyethylene glycol (PEG). It is believed that the polyethylene glycol acts to fuse the cell membranes, thus permitting the contents of the medium to be delivered into the cytoplasm of the Trichoderma sp. strain and the plasmid DNA is transferred to the nucleus. This fusion frequently leaves multiple copies of the plasmid DNA integrated into the host chromosome.

Usually a suspension containing the Trichoderma sp. protoplasts or cells that have been subjected to a permeability treatment at a density of 10⁵ to 10⁷/mL, typically, 2×10⁶/mL are used in transformation. A volume of 100 μL of these protoplasts or cells in an appropriate solution (e.g., 1.2 M sorbitol; 50 mM CaCl₂) are mixed with the desired DNA. Generally, a high concentration of PEG may be added to the uptake solution. From 0.1 to 1 volume of 25% PEG 4000 can be added to the protoplast suspension. It is also typical to add about 0.25 volumes to the protoplast suspension. Additives such as dimethyl sulfoxide, heparin, spermidine, potassium chloride and the like may also be added to the uptake solution and aid in transformation. Similar procedures are available for other fungal host cells. See, e.g., U.S. Pat. Nos. 6,022,725 and 6,268,328.

Generally, the mixture can be then incubated at approximately 0° C. for a period of between 10 to 30 minutes. Additional PEG may then be added to the mixture to further enhance the uptake of the desired gene or DNA sequence. The 25% PEG 4000 can be generally added in volumes of 5 to 15 times the volume of the transformation mixture; however, greater and lesser volumes may be suitable. The 25% PEG 4000 may be typically about 10 times the volume of the transformation mixture. After the PEG is added, the transformation mixture can then be incubated either at room temperature or on ice before the addition of a sorbitol and CaCl₂ solution. The protoplast suspension can then be further added to molten aliquots of a growth medium. This growth medium permits the growth of transformants only.

Generally, cells are cultured in a standard medium containing physiological salts and nutrients (see, e.g., Pourquie, J. et al., BIOCHEMISTRY AND GENETICS OF CELLULOSE DEGRADATION, eds. Aubert, J. P. et al., Academic Press, pp. 7186, 1988 and IImen, M. et al., (1997) Appl. Environ. Microbiol. 63:1298-1306). Common commercially prepared media (e.g., Yeast Malt Extract (YM) broth, Luria Bertani (LB) broth and Sabouraud Dextrose (SD) broth also find use in the present embodiments.

Culture-conditions are also standard, (e.g., cultures are incubated at approximately 28° C. in appropriate medium in shake cultures or fermentors until desired levels of glucoamylase expression are achieved). After fungal growth has been established, the cells are exposed to conditions effective to cause or permit the expression of the glucoamylase. In cases where the glucoamylase coding sequence is under the control of an inducible promoter, the inducing agent (e.g., a sugar, metal salt or antimicrobial), can be added to the medium at a concentration effective to induce glucoamylase expression.

In general, the glucoamylase produced in cell culture may be secreted into the medium and may be purified or isolated, e.g., by removing unwanted components from the cell culture medium. In some cases, the glucoamylase can be produced in a cellular form, necessitating recovery from a cell lysate. In such cases, the enzyme may be purified from the cells in which it was produced using techniques routinely employed by those of skill in the art. Examples of these techniques include, but are not limited to, affinity chromatography (Tilbeurgh et al., (1984) FEBS Lett. 16: 215), ion-exchange chromatographic methods (Goyal et al., (1991) Biores. Technol. 36: 37; Fliess et al., (1983) Eur. J. Appl. Microbiol. Biotechnol. 17: 314; Bhikhabhai et al, (1984) J. Appl. Biochem. 6: 336; and Ellouz et al., (1987) Chromatography 396: 307), including ion-exchange using materials with high resolution power (Medve et al., (1998) J. Chromatography A 808: 153), hydrophobic interaction chromatography (see, Tomaz and Queiroz, (1999) J. Chromatography A 865: 123; two-phase partitioning (see, Brumbauer, et al., (1999) Bioseparation 7: 287); ethanol precipitation; reverse phase HPLC, chromatography on silica or on a cation-exchange resin such as DEAE, chromatofocusing, SDS-PAGE, ammonium sulfate precipitation, and gel filtration (e.g., Sephadex G-75).

2.2. Alpha-Amylases

Alpha-amylases constitute a group of enzymes present in microorganisms and tissues from animals and plants. They are capable of hydrolyzing alpha-1,4-glucosidic bonds of glycogen, starch, related polysaccharides, and some oligosaccharides. Although all alpha-amylases possess the same catalytic function, their amino acid sequences vary greatly. The sequence identity between different amylases can be virtually non-existent, e.g., falling below 25%. Despite considerable amino acid sequence variation, alpha-amylases share a common overall topological scheme that has been identified after the three-dimensional structures of alpha-amylases from different species have been determined. The common three-dimensional structure reveals three domains: (1) a “TIM” barrel known as domain A, (2) a long loop region known as domain B that is inserted within domain A, and (3) a region close to the C-terminus known as domain C that contains a characteristic beta-structure with a Greek-key motif.

“Termamyl-like” alpha-amylases refer to a group of alpha-amylases widely used in the starch-processing industry. The Bacillus licheniformis alpha-amylase having an amino acid sequence of SEQ ID NO: 2 of U.S. Pat. No. 6,440,716 is commercially available as Termamyl®. Termamyl-like alpha-amylases commonly refer to a group of highly homologous alpha-amylases produced by Bacillus spp. Other members of the group include the alpha-amylases from Geobacillus stearothermophilus (previously known as Bacillus stearothermophilus; both names are used interchangeably in the present disclosure) and Bacillus amyloliquefaciens, and those derived from Bacillus sp. NCIB 12289, NCIB 12512, NCIB 12513, and DSM 9375, all of which are described in detail in U.S. Pat. No. 6,440,716 and WO 95/26397.

Although alpha-amylases universally contain the three domains discussed above, the three-dimensional structures of some alpha-amylases, such as AmyE from Bacillus subtilis, differ from Termamyl-like alpha-amylases. These enzymes are collectively referred as non-Termamyl-like alpha-amylases. “AmyE” for the purpose of this disclosure means a naturally occurring alpha-amylase (EC 3.2.1.1; 1,4-α-D-glucan glucanohydrolase) from Bacillus subtilis. Representative AmyE enzymes and the variants thereof are disclosed in U.S. patent application Ser. No. 12/478,266 and Ser. No. 12/478,368, both filed Jun. 4, 2009, and Ser. No. 12/479,427, filed Jun. 5, 2009.

Other commercially available amylases can be used, e.g., TERMAMYL® 120-L, LC and SC SAN SUPER®, SUPRA®, and LIQUEZYME® SC available from Novo Nordisk A/S, FUELZYME® FL from Diversa, and CLARASE® L, SPEZYME® FRED, SPEZYME® ETHYL, GC626, and GZYME® G997 available from Danisco, US, Inc., Genencor Division.

2.3. Other Enzymes and Enzyme Combinations

In embodiments of the present disclosure, other enzyme(s) may also be supplemented in starch processing, during saccharification and/or fermentation. These supplementary enzymes may include proteases, pullulanases, isoamylases, cellulases, hemicellulases, xylanases, cyclodextrin glycotransferases, lipases, phytases, laccases, oxidases, esterases, cutinases, xylanases, and/or alpha-glucosidases. See e.g., WO 2009/099783. Skilled artisans in the art are well aware of the methods using the above-listed enzymes.

The glucoamylases disclosed herein can be used in combination with any other enzyme. For example, glucoamylase maybe used in combination with amylases (e.g., alpha-amylases). In one embodiment, saccharification and/or fermentation or the simultaneous saccharification and fermentation (SSF) process use glucoamylase and one or more non-starch polysaccharide hydrolyzing enzymes. These enzymes are capable of hydrolyzing complex carbohydrate polymers such as cellulose, hemicellulose, and pectin. Non-limiting examples include cellulases (e.g., endo and exo-glucanases, beta glucosidase) hemicellulases (e.g., xylanases) and pectinases. In another embodiment, saccharification and/or fermentation or the SSF process use glucoamylase, alpha-amylase and one or more non-starch polysaccharide hydrolyzing enzymes. In another embodiment, saccharification and/or fermentation or the SSF process use glucoamylase with phytases, proteases, isoamylases and pullulanases.

In some embodiments, the saccharification and/or fermentation or the SSF process can use at least two non-starch polysaccharide hydrolyzing enzymes. In some embodiments, the saccharification and/or fermentation or the SSF process can use at least three non-starch polysaccharide hydrolyzing enzymes.

Cellulases are enzyme compositions that hydrolyze cellulose (β-1,4-D-glucan linkages) and/or derivatives thereof, such as phosphoric acid swollen cellulose. Cellulases include the classification of exo-cellobiohydrolases (CBH), endoglucanases (EG) and β-glucosidases (BG) (EC3.2.191, EC3.2.1.4 and EC3.2.1.21). Examples of cellulases include cellulases from Penicillium, Trichoderma, Humicola, Fusarium, Thermomonospora, Cellulomonas, Hypocrea, Clostridium, Thermomonospore, Bacillus, Cellulomonas and Aspergillus. Non-limiting examples of commercially available cellulases sold for feed applications are beta-glucanases such as ROVABIO® (Adisseo), NATUGRAIN® (BASF), MULTIFECT® BGL (Danisco Genencor) and ECONASE® (AB Enzymes). Some commercial cellulases includes ACCELERASE®. The cellulases and endoglucanases described in US20060193897A1 also may be used.

Beta-glucosidases (cellobiase) hydrolyzes cellobiose into individual monosaccharides. Various beta glucanases find use in the invention in combination with phytases. Beta glucanases (endo-cellulase-enzyme classification EC 3.2.1.4) also called endoglucanase I, II, and III, are enzymes that will attack the cellulose fiber to liberate smaller fragments of cellulose which is further attacked by exo-cellulase to liberate glucose. Commercial beta-glucanases useful in the methods of the invention include OPTIMASH® BG and OPTIMASH® TBG (Danisco, US, Inc. Genencor Division).

Hemicellulases are enzymes that break down hemicellulose. Hemicellulose categorizes a wide variety of polysaccharides that are more complex than sugars and less complex than cellulose, that are found in plant walls. In some embodiments, a xylanase find use as a secondary enzyme in the methods of the invention. Any suitable xylanase can be used in the invention. Xylanases (e.g. endo-β-xylanases (E.C. 3.2.1.8), which hydrolyze the xylan backbone chain, can be from bacterial sources (e.g., Bacillus, Streptomyces, Clostridium, Acidothermus, Microtetrapsora or Thermonospora) or from fungal sources (Aspergillus, Trichoderma, Neurospora, Humicola, Penicillium or Fusarium (See, e.g., EP473 545; U.S. Pat. No. 5,612,055; WO 92/06209; and WO 97/20920)). Xylanases useful in the invention include commercial preparations (e.g., MULTIFECT® and FEEDTREAT® Y5 (Danisco Genencor), RONOZYME®WX (Novozymes A/S) and NATUGRAIN WHEAT® (BASF). In some embodiments the xylanase is from Trichoderma reesei or a variant xylanase from Trichoderma reesei, or the inherently thermostable xylanase described in EP1222256B1, as well as other xylanases from Aspergillus niger, Aspergillus kawachii, Aspergillus tubigensis, Bacillus circulans, Bacillus pumilus, Bacillus subtilis, Neocallimastix patriciarum, Penicillium species, Streptomyces lividans, Streptomyces thermoviolaceus, Thermomonospora fusca, Trichoderma harzianum, Trichoderma reesei, and Trichoderma viridae.

Phytases that can be used include those enzymes capable of liberating at least one inorganic phosphate from inositol hexaphosphate. Phytases are grouped according to their preference for a specific position of the phosphate ester group on the phytate molecule at which hydrolysis is initiated, (e.g., as 3-phytases (EC 3.1.3.8) or as 6-phytases (EC 3.1.3.26)). A typical example of phytase is myo-inositol-hexakiphosphate-3-phosphohydrolase. Phytases can be obtained from microorganisms such as fungal and bacterial organisms (e.g. Aspergillus (e.g., A. niger, A. terreus, and A. fumigatus), Myceliophthora (M. thermophila), Talaromyces (T. thermophilus) Trichoderma spp (T. reesei). And Thermomyces (See e.g., WO 99/49740)). Also phytases are available from Penicillium species, (e.g., P. hordei (See e.g., ATCC No. 22053), P. piceum (See e.g., ATCC No. 10519), or P. brevi-compactum (See e.g., ATCC No. 48944) (See, e.g. U.S. Pat. No. 6,475,762). Additional phytases that find use in the invention are available from Peniophora, E. coli, Citrobacter, Enterbacter and Buttiauxella (see e.g., WO2006/043178, filed Oct. 17, 2005). Additional phytases useful in the invention can be obtained commercially (e.g. NATUPHOS® (BASF), RONOZYME® P (Novozymes A/S), PHZYME® (Danisco A/S, Diversa) and FINASE® (AB Enzymes).

Various acid fungal proteases (AFP) can be used as part of the combination as well. Acid fungal proteases include for example, those obtained from Aspergillus, Trichoderma, Mucor and Rhizopus, such as A. niger, A. awamori, A. oryzae and M. miehei. AFP can be derived from heterologous or endogenous protein expression of bacteria, plants and fungi sources. IAFP secreted from strains of Trichoderma can be used. Suitable AFP includes naturally occurring wild-type AFP as well as variant and genetically engineered mutant AFP. Some commercial AFP enzymes useful in the invention include FERMGEN® (Danisco US, Inc, Genencor Division), and FORMASE® 200.

Proteases can also be used with glucoamylase and any other enzyme combination. Any suitable protease can be used. Proteases can be derived from bacterial or fungal sources. Sources of bacterial proteases include proteases from Bacillus (e.g., B. amyloliquefaciens, B. lentus, B. licheniformis, and B. subtilis). Exemplary proteases include, but are not limited to, subtilisin such as a subtilisin obtainable from B. amyloliquefaciens and mutants thereof (U.S. Pat. No. 4,760,025). Suitable commercial protease includes MULTIFECT® P 3000 (Danisco Genencor) and SUMIZYME® FP (Shin Nihon). Sources of suitable fungal proteases include, but are not limited to, Trichoderma, Aspergillus, Humicola and Penicillium, for example.

Debranching enzymes, such as an isoamylase (EC 3.2.1.68) or pullulanase (EC 3.2.1.41), can also be used in combination with the glucoamylases in the saccharification and/or fermentation or SSF processes of the invention. A non-limiting example of a pullulanase that can be used is Promozyme®.

### 3. Starch Processing

3.1. Starch Substrates and Raw Materials

Those of skill in the art are well aware of available methods that may be used to prepare starch substrates for use in the processes disclosed herein. For example, a useful starch substrate may be obtained from tubers, roots, stems, legumes, cereals, or whole grain. More specifically, the granular starch comes from plants that produce high amounts of starch. For example, granular starch may be obtained from corn, wheat, barley, rye, milo, sago, cassava, tapioca, sorghum, rice, peas, bean, banana, or potatoes. Corn contains about 60-68% starch; barley contains about 55-65% starch; millet contains about 75-80% starch; wheat contains about 60-65% starch; and polished rice contains about 70-72% starch. Specifically contemplated starch substrates are cornstarch, wheat starch, and barley starch. The starch from a grain may be ground or whole and includes corn solids, such as kernels, bran and/or cobs. The starch may be highly refined raw starch or feedstock from starch refinery processes. Various starches also are commercially available. For example, cornstarch may be available from Cerestar, Sigma, and Katayama Chemical Industry Co. (Japan); wheat starch may be available from Sigma; sweet potato starch may be available from Wako Pure Chemical Industry Co. (Japan); and potato starch may be available from Nakaari Chemical Pharmaceutical Co. (Japan).

3.2. Milling

The starch substrate can be a crude starch from milled whole grain, which contains non-starch fractions, e.g., germ residues and fibers. Milling may comprise either wet milling or dry milling. In wet milling, whole grain can be soaked in water or dilute acid to separate the grain into its component parts, e.g., starch, protein, germ, oil, kernel fibers. Wet milling efficiently separates the germ and meal (i.e., starch granules and protein) and can be especially suitable for production of syrups. In dry milling, whole kernels are ground into a fine powder and processed without fractionating the grain into its component parts. Dry milled grain thus will comprise significant amounts of non-starch carbohydrate compounds, in addition to starch. Most ethanol comes from dry milling. Alternatively, the starch to be processed may be a highly refined starch quality, for example, at least about 90%, at least about 95%, at least about 97%, or at least about 99.5% pure.

3.3. Gelatinization and Liquefaction

In some embodiments of the invention, gelatinazation and/or liquefaction may be used. As used herein, the term “liquefaction” or “liquefy” means a process by which starch is converted to less viscous and soluble shorter chain dextrins. In some embodiments, this process involves gelatinization of starch simultaneously with or followed by the addition of alpha-amylases. Additional liquefaction-inducing enzymes, e.g., a phytase, optionally may be added. In some embodiments, gelatinization is not used. In other embodiments, a separate liquefaction step is not used. Starches can be converted to shorter chains at the same time that saccharification and/or fermentation is performed. In some embodiments, the starch is being converted directly to glucose. In other embodiments, a separate liquefaction step is used prior to saccharification.

In some embodiments, the starch substrate prepared as described above may be slurried with water. The starch slurry may contain starch as a weight percent of dry solids of about 10-55%, about 20-45%, about 30-45%, about 30-40%, or about 30-35%. In some embodiments, the starch slurry is at least about 5%, at least about 10%, at least about 15%, at least about 20%, at least about 25%, at least about 30%, at least about 35%, at least about 40%, at least about 45%, at least about 50%, or at least about 55%.

To optimize alpha-amylase stability and activity, the pH of the slurry may be adjusted to the optimal pH for the alpha-amylases. Alpha-amylases remaining in the slurry following liquefaction may be deactivated by lowering pH in a subsequent reaction step or by removing calcium from the slurry. The pH of the slurry should be adjusted to a neutral pH (e.g., pH 5.0 to 8.0 and any pH in between this range) when the glucoamylases of the invention are used.

The slurry of starch plus the alpha-amylases may be pumped continuously through a jet cooker, which may be steam heated from about 85° C. to up to about 105° C. Gelatinization occurs very rapidly under these conditions, and the enzymatic activity, combined with the significant shear forces, begins the hydrolysis of the starch substrate. The residence time in the jet cooker can be very brief. The partly gelatinized starch may be passed into a series of holding tubes maintained at about 85-105° C. and held for about 5 min. to complete the gelatinization process. These tanks may contain baffles to discourage back mixing. As used herein, the term “secondary liquefaction” refers the liquefaction step subsequent to primary liquefaction, when the slurry is allowed to cool to room temperature. This cooling step can be about 30 minutes to about 180 minutes, e.g., about 90 minutes to 120 minutes. Milled and liquefied grain is also known as mash.

3.4. Saccharification

Following liquefaction, the mash can be further hydrolyzed through saccharification to produce fermentable sugars that can be readily used in the downstream applications. The saccharification of the present embodiments can be carried out at a pH in the range of 5.0 to 8.0, 5.5 to 7.5, 6.0 to 7.5, 6.5 to 7.5, or 7.0 to 7.5, by using a glucoamylase as described above. In other embodiments, the pH used can be 5.0, 5.25, 5.50, 5.75, 6.0, 6.50, 7.0, 7.50 or 8.0.

In one embodiment, at pH 6.0 or higher, the glucoamylase possesses at least about 50%, about 51%, about 52%, about 53%, about 54%, or about 55% activity relative to its maximum activity at the optimum pH. In another embodiment, for a pH range of 6.0 to 7.5, HgGA can have at least 53% activity relative to its maximum activity. In another embodiment, at pH 6.0, TrGA can have at least 50% activity relative to its maximum activity. In one embodiment, a glucoamylase (e.g. HgGA) has 67% maximal activity at pH 7.0. In another embodiment, a glucoamylase (e.g., TrGA) has 66% maximal activity at pH 5.25.

In one embodiment, the glucoamylase may be dosed at the range of about 0.2 to 2.0 GAU/g dss, about 0.5 to 1.5 GAU/g dss, or 1.0 to 1.5 GAU/g dss. In one embodiment, glucoamylase (e.g., TrGA) can be used at a dose of about 1 to 5 GAU/gds starch. In another embodiment, glucoamylase (e.g., TrGA) can be used at a dose of about 1 GAU/gds starch, 2 GAU/gds starch, 3 GAU/gds starch, 4 GAU/gds starch, or 5 GAU/gds starch. In one embodiment, glucoamylase (e.g., HgGA) can be used at a dose of about 0.25 to 1 GAU/gds starch. In another embodiment, glucoamylase (e.g., HgGA) can be used at a dose of about 0.25 GAU/gds starch, 0.5 GAU/gds starch, 0.75 GAU/gds starch, or 1 GAU/gds starch. The saccharification may be performed at about 30 to about 60° C., or about 40 to about 60° C. In some embodiments, the saccharification occurs at ph 7.0 at 32° C. In other embodiments, the saccharification occurs at ph 6.5 at 58° C.

A full saccharification step may typically range 24 to 96 hours, 24 to 72 hours, or 24 to 48 hours. In some embodiments, saccharification occurs after about 2, 4, 6, 7.7, 8, 110, 14, 16, 18, 20, 22, 23.5, 24, 26, 28, 30, 31.5, 34, 36, 38, 40, 42, 44, 46, or 48 hours. In some embodiments, the saccharification step and fermentation step are combined and the process is referred to as simultaneous saccharification and fermentation (SSF).

It is understood that generally, as time elapses, the enzymes (glucoamylase with or without other enzymes, such as alpha-amylases or non-starch polysaccharide hydrolyzing enzyme) reduces the higher sugars to lower DP sugars (such as DP1). The sugar profile can be varied by using different parameters, such as, but not limited to, starting starch substrate, temperature, amount of glucoamylase, type of glucoamylase, and pH. For example, in one embodiment, at 32 degrees Celsius and pH 7.0, the sugar or oligosaccharide distribution during the saccharification process can be between about 0.36% to about 96.50% DP1, about 3.59% to about 11.80% DP2, about 0.12% to about 7.75%, and/or about 2.26% to about 88.30% for higher sugars for HgGA. In another embodiment, at 32 degrees Celsius and pH 7.0, the sugar distribution during the saccharification process can be between about 0.36% to about 79.19% DP1, between about 3.59% to about 9.92% DP2, about 0.17% to about 9.10% DP3 and/or about 17.15% to about 88.30% for higher sugars for TrGA. Thus, in one embodiment, using HgGA, the DP1 content can reach more than 90% after 24 hours. After 45 hours, the DP1 content can reach more than 96%, while the content of higher sugars can decrease to less than 3%. Using TrGA, more than 70% DP1 can be obtained after 24 hours. After 45 hours, the DP1 content can reach about 80%, while the content of higher sugars can drop to less than 20%.

In another embodiment, at 58 degrees Celsius and pH 6.5, the sugar distribution during the saccharification process can be between about 60.66% to about 93.67% DP1, between about 1.49% to about 8.87% DP2, about 0.33% to about 1.93% DP3 and/or about 4.51% to about 28.17% for higher sugars for HgGA. In other embodiments, at 58 degrees Celsius and pH 6.5, the sugar or oligosaccharide distribution during the saccharification process can be between about 37.08% to about 75.25% DP1, about 5.48% to about 10.19% DP2, about 0.46% to about 5.06%, and/or about 18.37% to about 47.47% for higher sugars for TrGA. Thus, in one embodiment, using HgGA, the DP1 content can reach more than 90% after 24 hours. After 48 hours, the DP1 content can reach more than 93%, while the content of higher sugars can decrease to less than 5%. Using TrGA, more than 70% DP1 can be obtained after 24 hours. After 45 hours, the DP1 content can reach about 75%, while the content of higher sugars can drop to about 18%.

In yet another embodiment, at 58 degrees Celsius and pH 6.5, glucoamylases disclosed herein can be used to saccharify a starch substrate where high sugars (e.g., DP4+) is reduced. In some embodiments, the sugar or oligosaccharide distribution during the saccharification process can be between about 81.10% to about 90.36% DP1, about 1.99% to about 3.96% DP2, about 0.49% to about 0.61% DP3, about 4.48% to about 16.13% DP4+ for TrGA. In other embodiments, the sugar or oligosaccharide distribution during the saccharification process can be between about 93.15% to about 95.33% DP1, about 2.10% to about 3.94% DP2, about 0.53% to about 1.00% DP3, about 0.94% to about 3.76% DP4+ for HgGA.

In yet another embodiment, at 58 degrees Celsius and pH 6.4, the sugar or oligosaccharide distribution during the saccharification process can be between about 93.79% to about 96.9% DP1, about 1.55% to about 3.02% DP2, about 0.2% to about 0.49% DP3 and about 0% to about 3.98% DP4+ for HgGA. In some cases, about 93% solubility and about 96.9% glucose yield can be achieved within 24 hours. Continuous saccharification can result in 99% solubility and about 96.8% glucose after about 48 hours.

In another embodiment, at 58 degrees Celsius and pH 6.4, the sugar or oligosaccharide distribution during the saccharification process can be between about 75.08% to about 96.5% DP1, 1.57% to about 9.16% DP2, 0.67% to about 15.76% DP3+. In some cases, HgGA can maintain a significant amount of glucoamylase activity for about 52 hours at pH6.4 to yield continued production of DP1 products, DP2 products, and increase of percentage of soluble solids. Increased amounts of HgGA can result in increased rates of percentage solubilization and DP1 production.

3.5. Fermentation

In some embodiments of the present disclosure, the fermentable sugars may be subject to batch or continuous fermentation conditions. A classical batch fermentation is a closed system, wherein the composition of the medium is set at the beginning of the fermentation and is not subject to artificial alterations during the fermentation. Thus, at the beginning of the fermentation the medium may be inoculated with the desired organism(s). In this method, fermentation can be permitted to occur without the addition of any components to the system. Typically, a batch fermentation qualifies as a “batch” with respect to the addition of the carbon source and attempts are often made at controlling factors such as pH and oxygen concentration. The metabolite and biomass compositions of the batch system change constantly up to the time the fermentation is stopped. Within batch cultures, cells progress through a static lag phase to a high growth log phase, and finally to a stationary phase where growth rate is diminished or halted. If untreated, cells in the stationary phase eventually die. In general, cells in log phase are responsible for the bulk of production of the end product.

A variation on the standard batch system is the “fed-batch fermentation” system, which may be used in some embodiments of the present disclosure. In this variation of a typical batch system, the substrate can be added in increments as the fermentation progresses. Fed-batch systems are particularly useful when catabolite repression is apt to inhibit the metabolism of the cells and where it is desirable to have limited amounts of substrate in the medium. Measurement of the actual substrate concentration in fed-batch systems may be difficult and is therefore estimated on the basis of the changes of measurable factors such as pH, dissolved oxygen and the partial pressure of waste gases such as CO₂. Both batch and fed-batch fermentations are common and well known in the art.

On the other hand, continuous fermentation is an open system where a defined fermentation medium can be added continuously to a bioreactor and an equal amount of conditioned medium can be removed simultaneously for processing. Continuous fermentation generally maintains the cultures at a constant high density where cells are primarily in log phase growth. Continuous fermentation allows for the modulation of one factor or any number of factors that affect cell growth and/or end product concentration. For example, in one embodiment, a limiting nutrient such as the carbon source or nitrogen source can be maintained at a fixed rate while all other parameters are allowed to moderate. In other systems, a number of factors affecting growth can be altered continuously while the cell concentration, measured by media turbidity, may be kept constant. Continuous systems strive to maintain steady state growth conditions. Thus, cell loss due to medium being drawn off must be balanced against the cell growth rate in the fermentation. Methods of modulating nutrients and growth factors for continuous fermentation processes as well as techniques for maximizing the rate of product formation are well known in the art of industrial microbiology.

In further embodiments, by use of appropriate fermenting microorganisms as known in the art, the fermentation end product may include without limitation alcohol, 1,3-propanediol, succinic acid, lactic acid, amino acids, proteins, functional oligosaccharides, and derivatives thereof. See e.g., WO 2008/086811 (methanol, ethanol, propanol, and butanol fermentation); WO 2003/066816, U.S. Pat. Nos. 5,254,467 and 6,303,352 (1,3-propanediol fermentation); U.S. Pat. Nos. RE 37,393, 6,265,190, and 6,596,521 (succinic acid fermentation); U.S. Pat. No. 5,464,760, WO 2003/095659, Mercier et al., J. Chem. Tech. Biotechnol. 55: 111-121, Zhang and Cheryan, Biotechnol. Lett. 13: 733-738 (1991), Linko and Javanainen, Enzyme Microb. Technol. 19: 118-123 (1996), and Tsai and Moon, Appl. Biochem. Biotechnol. 70-72: 417-428 (1998) (lactic acid fermentation); U.S. Pat. Nos. 7,320,882, 7,332,309, 7,666,634, and Zhang et al., Appl. Microbiol. Biotechnol. 77: 355-366 (2007) (fermentation of various amino acids). The above enumerated list are only examples and one skilled in the art will be aware of a number of fermenting microorganisms that may be appropriately used to obtain a desired end product.

3.6. Simultaneous Saccharification and Fermentation (SSF)

During SSF, the hydrolyzing enzymes are added along with the end product producer, commonly a microorganism. Enzymes release fermentable lower molecular weight sugars, i.e., fermentable sugars DP1-3, from the starch substrate, while the microorganism simultaneously uses the fermentable sugars for growth and production of the end product. Typically, fermentation conditions are selected that provide an optimal pH and temperature for promoting the best growth kinetics of the producer host cell strain and catalytic conditions for the enzymes produced by the culture. See e.g., Doran et al., Biotechnol. Progress 9: 533-538 (1993). Table 1 presents exemplary fermentation microorganism and their optimal pH for fermentation. Because the glucoamylases disclosed herein possess significant activity at a neutral pH and an elevated temperature, they would be useful in the SSF for those microorganisms having an optimal fermenting pH in the range of 5.5 to 7.5.

TABLE 1
Exemplary fermentation organisms and their optimal pH.

| | | Optimal pH of the |
|---|---|---|
| End products | Fermentation Organisms | fermentation |
| Lysine and salts | Corynebacterium glutamicum | 6.8-7.0 |
| thereof | Bacillus lacterosprous | 7.0-7.2 |
| | Methylophilotrophus | 7 |
| Lactic Acid | Lactobacillus amylophilus | 6.0-6.5 |
| | Bacillus coagulans | 6.4-6.6 |
| | Bacillus thermoamylovorans | 5.0-6.5 |
| | Bacillus smithii | 5.0-6.5 |
| | Geobacillus stearothermophilus | 5.0-6.5 |
| Monosodium | Corynebacterium pekinense | 7 |
| Glutamate (MSG) | Corynebacterium crenatum | 7 |
| | Brevibacterium tianjinese | 7 |
| | Corynebacterium glutamicum | 7.0-7.2 |
| | HU7251 | |
| | Arthrobacter sp | 7 |
| Succinic acid | Escherichia coli | 6.0-7.5 |
| 1,3-Propanediol | Escherichia coli | 6.5-7.5 |
| 2-Keto-gulonic acid | Escherichia coli | 5.0-6.0 |

In further embodiments, by use of appropriate fermenting microorganisms as known in the art to produce the desired end product, those of skill in the art are well capable of adjusting the SSF conditions, e.g., temperature, nutrient composition, light conditions, oxygen availability, etc.

### 4. Methods Used in the Examples

The following materials, assays, and methods are used in the examples provided below:

HPLC Method to Measure Saccharide Composition

The composition of the reaction products of oligosaccharides was measured by a HPLC system (Beckman System Gold 32 Karat Fullerton, Calif.). The system, maintained at 50° C., was equipped with a Rezex 8 u8% H Monosaccharides column and a refractive index (RI) detector (ERC-7515A, Anspec Company, Inc.). Diluted sulfuric acid (0.01 N) was applied as the mobile phase at a flow rate of 0.6 ml/min. 20 μl of 4.0% solution of the reaction mixture was injected onto the column. The column separates saccharides based on their molecular weights. The distribution of saccharides and the amount of each saccharide were determined from previously run standards.

Determination of Glucoamylase Activity Units (GAU)

Glucoamylase activity units (GAU) were determined based on the activity of a glucoamylase enzyme to catalyze the hydrolysis of p-nitrophenyl-alpha-D-glucopyranoside (PNPG) to glucose and p-nitrophenol. At an alkaline pH, p-nitrophenol forms a yellow color that is measured spectrophotometrically at 405 nm. The amount of p-nitrophenol released correlates with the glucoamylase activity.

Protein Concentration Determination

The protein concentration in a sample was determined using the Bradford QuickStart™ Dye Reagent (Bio-Rad, California, USA). For example, a 10 μL sample of the enzyme was combined with 200 μL Bradford QuickStart™ Dye Reagent. After thorough mixing, the reaction mixture was incubated for at least 10 minutes at room temperature. Air bubbles were removed and the optical density (OD) was measured at 595 nm. The protein concentration was then calculated using a standard curve generated from known amounts of bovine serum albumin.

Purification of HgGA for Characterization Studies

The material concentrated by ultrafiltration (UFC) was desalted/buffer-exchanged using a BioRad DP-10 desalting column and 25 mM Tris pH 8.0. 100 mg of total protein was applied to a Pharmacia Hi Prep 16/10 S Sepharose FF column, which was equilibrated with the above buffer at 5 ml/min. Glucoamylase was eluted with a 4-column volume (CV) gradient buffer containing 0-200 mM NaCl. Multiple runs were performed and the purest fractions, as determined via SDS-PAGE/coomassie blue staining analysis, were pooled and concentrated using VivaSpin 10K MWCO 25 ml spin tubes. The final material was passed over a Novagen HisBind 900 chromatography cartridge that had been washed with 250 mM EDTA and rinsed with above buffer. 2 ml of final material was obtained, having a protein concentration of 103.6 mg/ml, and a glucoamylase activity of 166.1 GAU/ml (determined by a PNPG based assay). Specific activities were determined using a standardized method using p-nitrophenyl-alpha-D-glucopyranoside (PNPG) as a substrate and reported in GAU units.

Determination of Glucose Concentration

Glucose concentration in a saccharification reaction mixture was determined with the ABTS assay. Samples or glucose standards in 5 μL were placed in wells of a 96-well microtiter plate (MTP). Reactions were initiated with the addition of 95 μL of the reactant containing 2.74 mg/ml 2,2′-Azino-bis(3-ethylbenzothiazoline-6-sulfonic acid)diammonium salt (ABTS) (Sigma P1888), 0.1 U/ml horseradish peroxidase type VI (Sigma P8375), and 1 U/ml glucose oxidase (Sigma G7141). OD_{405 nm} was immediately monitored at a 9-second interval for 300 seconds using a Spectramax plate reader. Because the rate of OD_{405 nm} increase is proportional to the glucose concentration, the sample's glucose concentration was determined by comparing with the glucose standard, and was reported as mg/ml.

Lactic Acid Fermentation Conditions

Strains of Lactobacillus rhamnosus and Bacillus coagulans were obtained from China General Microbiological Culture Collection Center.

Seed media (MRS 006): Casein 10.0 g, Beef extract 10.0 g, Yeast extract 5.0 g, Glucose 5.0 g, Sodium acetate 5.0 g, diammonium citrate 2.0 g, Tween® 80 1.0 g, K₂HPO₄ 2.0 g, MgSO₄.7H₂O 0.2 g, MnSO₄.H₂O 0.05 g, Distilled water 1.0 L, pH 6.8; additional 20 g agar were added for inoculum media.

Fermentation medium: corn steep liquor 40 g, casein 10.0 g, Beef extract 10.0 g, Yeast extract 10.0 g, cassava starch 150 g, Tween® 80 1.5 g, MnSO₄.H₂O 0.3 g, Calcium carbonate 20 g, Distilled water 1.0 L, pH 6.5. Starch (cassava starch or cornstarch) or glucose was added based on different test conditions.

Starch (cassava starch, cornstarch, or wheat starch), glucose, agar, corn steep liquor, casein, Beef extract, Yeast extract, Glucose, Sodium acetate, diammonium hydrogen citrate, Tween® 80, K₂HPO₄, MgSO₄.7H₂O, MnSO₄.H₂O, CaCO₃, and Agar powder were all of analytic grade and procured locally.

The inoculum of Lactobacillus rhamnosus was transferred to each 100 mL seed culture and cultivated at 37° C., 200 rpm, for 12 to 24 hrs, until OD₆₀₀ reached about 0.5. 10-20 mL seed culture was added to each fermentor with 1 L fermentation medium.

### EXAMPLES

### Example 1

### Comparison of the pH and Activity Profiles of Various Glucoamylases at 32° C

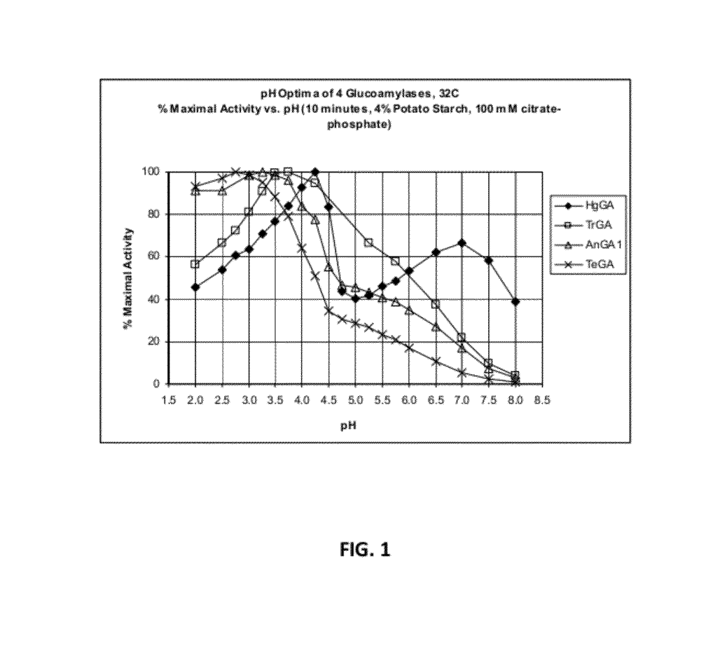

The pH and activity profiles of glucoamylases (GAs) from Humicola grisea (HgGA), Trichoderma reesei (TrGA), Aspergillus niger (AnGA) and Talaromyces emersonii (TeGA) were determined at 32° C. As the substrate, 8% potato starch (Sigma Cat. No. 52630) was solubilized by heating. A series of citrate/phosphate buffers at 0.25 or 0.5 pH increments, ranging from pH 2.0 to 8.0, were prepared. Purified enzymes were diluted to 0.1 or 0.02 GAU/ml in water (TeGA was dosed at 0.2 GAU/ml). HgGA, TrGA, AnGA, and TeGA were dosed at 0.0125, 0.0076, 0.0109, and 0.0055 mg/ml, respectively. 10 μL buffer of various pH was placed in 0.2 ml PCR tube strips (AB Gene, Cat. No. AB-0451, 800-445-2812) with 15 μL of diluted enzyme. The reactions were initiated by the addition of 25 μL soluble potato starch. The reactions were incubated on a PCR type thermocycler heating block for exactly ten minutes, then terminated by the addition of 10 μL 0.5 M NaOH. The glucose released in the reaction was determined using the ABTS assay, and the glucoamylase activities were determined. The pH and activity profiles are presented in Table and FIG. 1 as the percentage of the maximum activity for each glucoamylase.

TABLE 2
pH profiles of HgGA, TrGA, AnGA, and TeGA at 32° C. The values
represent % of the maximum activity for each enzyme.

| pH | HgGA | TrGA | AnGA | TeGA |
|---|---|---|---|---|
| 2.00 | 45 | 56 | 91 | 93 |
| 2.50 | 54 | 67 | 91 | 97 |
| 2.75 | 60 | 72 | | 100 |
| 3.00 | 63 | 81 | 98 | 98 |
| 3.25 | 71 | 91 | 100 | 95 |
| 3.50 | 77 | 99 | 99 | 88 |
| 3.75 | 84 | 100 | 96 | 79 |
| 4.00 | 93 | | 84 | 64 |
| 4.25 | 100 | 95 | 78 | 51 |
| 4.50 | 84 | | 55 | 34 |
| 4.75 | 44 | | 46 | 30 |
| 5.00 | 40 | | 45 | 29 |
| 5.25 | 42 | 66 | 43 | 27 |
| 5.50 | 46 | | 41 | 23 |
| 5.75 | 48 | 58 | 39 | 21 |
| 6.00 | 53 | 51 | 35 | 17 |
| 6.50 | 62 | 38 | 27 | 11 |
| 7.00 | 67 | 22 | 17 | 5 |
| 7.50 | 58 | 10 | 7 | 2 |
| 8.00 | 39 | 4 | 3 | 1 |

As shown in Table 2 and FIG. 1, both TeGA and AnGA exhibited significantly reduced activity in the pH range of 6.0 to 8.0. At a pH 5.0 or above, TeGA retained no more than 29% activity relative to its maximum activity. At a pH 6.0 or above, TeGA retained no more than 17% activity relative to its maximum activity. Similarly, at a pH 6.0 of 6.0 or above, AnGA displayed no more than 35% activity relative to its maximum activity. In the pH range of 6.0 to 7.5, HgGA retained at least 53% activity relative to its maximum activity. At pH 6.0, TrGA also displayed at least 50% activity relative to its maximum activity. The above observation suggests that both HgGA and TrGA may be suitable for producing fermentable sugars at a neutral pH range (as described herein for neutral pH glucoamylases) under fermentation conditions.

### Example 2

### Comparison of Hydrolysis of Solubilized Starch at 32° C., pH 7.0

The ability of various glucoamylases to hydrolyze solubilized starch substrate (liquefact) at a neutral pH was compared. Corn starch was liquefied by following a conventional high-temperature jet cooking process using CLEARFLOW™ AA to a liquefact of DE 12-15. Saccharification of the liquefact (25% DS) was carried out using TrGA, HgGA, and AnGA at 1.0 GAU/g ds at 32° C., pH 7.0. Samples were withdrawn at different time intervals during the saccharification and subject to HPLC analysis. The composition of the oligosaccharides is presented in Table 3.

TABLE 3
Composition of oligosaccharides in saccharification.

% Sugars, pH 7.0, 32° C.

| | | | | | Higher |
|---|---|---|---|---|---|
| GA | Time (hr) | DP1 | DP2 | DP3 | Sugars |
| HgGA | 0 | 0.36 | 3.59 | 7.75 | 88.30 |
| | 2 | 51.10 | 10.20 | 6.87 | 31.85 |
| | 5.25 | 64.90 | 11.80 | 0.13 | 23.13 |
| | 21.25 | 89.30 | 1.10 | 0.30 | 9.34 |
| | 25.25 | 91.20 | 0.98 | 0.23 | 7.61 |
| | 29.25 | 92.60 | 0.90 | 0.31 | 6.12 |
| | 45.25 | 96.50 | 1.15 | 0.12 | 2.26 |
| TrGA | 0 | 0.36 | 3.59 | 7.75 | 88.30 |
| | 2 | 38.06 | 7.49 | 9.10 | 45.35 |
| | 5.25 | 47.17 | 9.92 | 6.13 | 36.78 |
| | 21.25 | 69.43 | 8.33 | 0.17 | 22.07 |
| | 25.25 | 71.69 | 7.14 | 0.17 | 21.01 |
| | 29.25 | 73.57 | 6.16 | 0.18 | 20.09 |
| | 45.25 | 79.19 | 3.45 | 0.20 | 17.15 |
| AnGA | 0 | 0.36 | 3.59 | 7.75 | 88.30 |
| | 2 | 14.12 | 4.57 | 8.88 | 72.43 |
| | 5.25 | 28.38 | 8.01 | 10.30 | 53.31 |
| | 21.25 | 58.97 | 11.49 | 0.28 | 29.26 |
| | 25.25 | 60.94 | 10.53 | 0.28 | 28.25 |
| | 29.25 | 62.82 | 9.54 | 0.23 | 27.41 |
| | 45.25 | 74.14 | 4.08 | 0.24 | 21.54 |

Using HgGA, the DP1 content reached more than 90% after 24 hrs. After 45 hours, the DP1 content reached more than 96%, while the content of higher sugars decreased to less than 3%. Using TrGA, more than 70% DP1 was obtained after 24 hours. After 45 hours, the DP1 content reaches about 80%, while the content of higher sugars dropped to less than 20%. For AnGA, less than 75% of DP1 was obtained after 45 hours, while higher sugars remained more than 20%. The data in Table 3 indicate that both HgGA and TrGA are more effective than AnGA to hydrolyze solubilized starch to glucose, at a neutral pH.

### Example 3

### Comparison of Hydrolysis of Liquefied Starch at 58° C., pH 6.5

Corn starch liquefact (˜9.1DE) obtained by SPEZYME® FRED (Danisco US Inc., Genencor Division) treatment was adjusted to pH 6.5 with NaOH and equilibrated at a 58° C. water bath. AnGA (OPTIDEX™ L-400, Danisco US Inc., Genencor Division), TrGA, and HgGA were added at 0.5 GAU/g ds to each flask containing corn starch liquefact. Saccharification was carried out up to 48 hours with periodical sampling for HPLC analysis. 0.5 mL enzyme-deactivated sample was diluted with 4.5 ml of RO water. The diluted sample was then filtered through 0.45 μm Whatman filters and subject to HPLC analysis. The HPLC analysis was conducted as described in Methods used in the Examples. The composition of the oligosaccharides is presented in Table 4.

TABLE 4
Composition of oligosaccharides in saccharification.

Percent Sugar Composition

| | Hour | % DP1 | % DP2 | % DP3 | % HS |
|---|---|---|---|---|---|
| Liquefact | 0 | 0.49 | 3.02 | 5.52 | 90.98 |
| HgGA | 2 | 60.66 | 8.87 | 1.93 | 28.17 |
| | 4 | 69.92 | 7.43 | 0.69 | 21.75 |
| | 6 | 75.96 | 5.80 | 0.38 | 17.85 |
| | 7.7 | 77.56 | 5.15 | 0.47 | 16.35 |
| | 14 | 84.31 | 2.96 | 0.42 | 11.57 |
| | 23.5 | 88.70 | 2.20 | 0.43 | 8.67 |
| | 31.5 | 90.01 | 1.87 | 0.40 | 6.90 |
| | 48 | 93.67 | 1.49 | 0.33 | 4.51 |
| TrGA | 2 | 37.08 | 10.19 | 5.06 | 47.47 |
| | 4 | 49.25 | 12.12 | 2.12 | 36.42 |
| | 6 | 55.30 | 12.16 | 1.09 | 31.10 |
| | 7.7 | 58.06 | 11.74 | 0.76 | 29.12 |
| | 14 | 63.83 | 9.96 | 0.46 | 25.28 |
| | 23.5 | 68.52 | 8.18 | 0.53 | 22.77 |
| | 31.5 | 70.35 | 7.24 | 0.54 | 21.32 |
| | 48 | 75.25 | 5.48 | 0.50 | 18.37 |
| AnGA | 2 | 41.33 | 11.83 | 4.40 | 42.20 |
| | 4 | 50.08 | 12.95 | 1.60 | 35.04 |
| | 6 | 53.32 | 12.70 | 0.83 | 33.16 |
| | 7.7 | 54.80 | 12.41 | 0.62 | 31.91 |
| | 14 | 58.85 | 11.20 | 0.40 | 29.15 |
| | 23.5 | 61.70 | 10.44 | 0.46 | 27.41 |
| | 31.5 | 62.34 | 10.11 | 0.50 | 26.58 |
| | 48 | 64.23 | 9.83 | 0.59 | 25.01 |

Using HgGA, the DP1 content reached more than 90% after 24 hrs. After 48 hours, the DP1 content reached more than 93%, while the content of higher sugars decreased to less than 5%. Using TrGA, more than 70% DP1 was obtained after 24 hours. After 45 hours, the DP1 content reaches about 75%, while the content of higher sugars dropped to about 18%. For AnGA, less than 65% of DP1 was obtained after 45 hours, while higher sugars remained more than 25%. The data in Table 4 indicate that both HgGA and TrGA are more effective than AnGA, at a neutral pH and 58° C., to hydrolyze solubilized starch to glucose. This observation is consistent with data presented in Table 3, where saccharification was performed at 32° C.

### Example 4

### Comparison of High Sugars (DP4+) Reduction at 58° C., pH 6.5

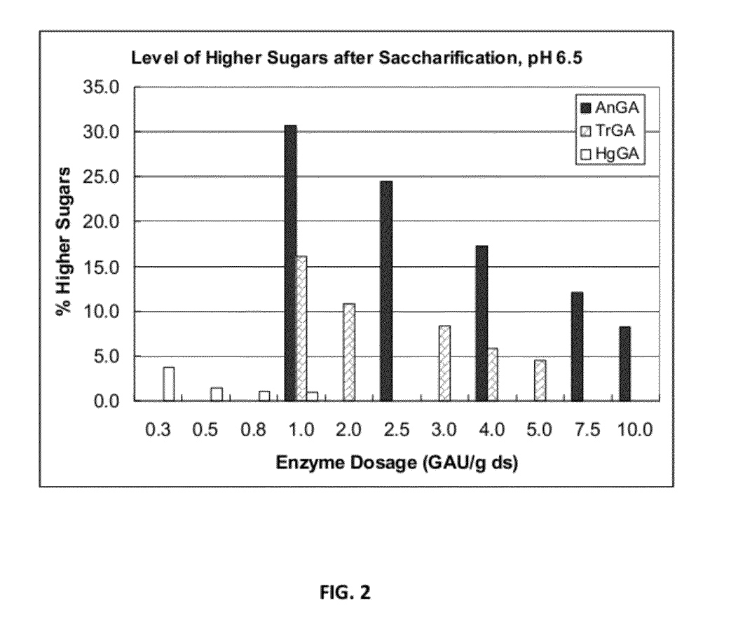

Various concentrations of AnGA, TrGA, and HgGA were used to saccharify a starch substrate at 58° C., pH 6.5, and the reduction of high sugars (DP4+) was compared. The starch substrate was a 25% cornstarch liquefact, which was liquefied by SPEZYME® FRED (Danisco US Inc., Genencor Division). Glucoamylases were added as shown in Table 5, from 0.25 GAU/gds to 10.0 GAU/gds. The saccharification reaction was conducted at 58° C., pH 6.5. Samples were withdrawn at various time points and the sugar composition was determined by HPLC analysis. The composition of the oligosaccharides is presented in Table 5 and FIG. 2.

TABLE 5
Composition of oligosaccharides in saccharification.

| | |
|---|---|
| | Percent Sugar Composition |
| GAU/gds | at 48 hr |

| Glucoamylase | starch | DP1 | DP2 | DP3 | DP4+ |
|---|---|---|---|---|---|
| AnGA | 1 | 64.25 | 5.10 | 0.00 | 30.65 |
| | 2.5 | 73.36 | 1.74 | 0.41 | 24.49 |
| | 5 | 81.26 | 1.05 | 0.46 | 17.22 |
| | 7.5 | 85.53 | 1.48 | 0.44 | 12.13 |
| | 10 | 89.32 | 2.03 | 0.42 | 8.22 |
| TrGA | 1 | 81.10 | 2.28 | 0.49 | 16.13 |
| | 2 | 86.65 | 1.99 | 0.49 | 10.87 |
| | 3 | 90.36 | 2.86 | 0.49 | 8.30 |
| | 4 | 90.48 | 3.17 | 0.52 | 5.83 |
| | 5 | 90.95 | 3.96 | 0.61 | 4.48 |
| HgGA | 0.25 | 93.15 | 2.10 | 1.00 | 3.76 |
| | 0.5 | 95.33 | 2.58 | 0.64 | 1.45 |
| | 0.75 | 95.08 | 3.36 | 0.53 | 1.02 |
| | 1 | 94.57 | 3.94 | 0.56 | 0.94 |

The results presented in Table 5 and FIG. 2 indicated that AnGA resulted in more than 8% of higher sugars (DP4+), at 58° C., pH 6.5, even at a high dosage of glucoamylase, 10.0 GAU/gds. In contrast, lower than 5% of higher sugars (DP4+) was observed for 5 GAU/gds TrGA. HgGA resulted in the lowest levels of higher sugars (DP4+). For example, at 0.5 GAU/gds HgGA, the saccharification mixture contained less than 1.5% of higher sugars (DP4+), which is comparable to the resulted obtained under the current industrial high glucose processing conditions (pH 4.5, 60° C.) using AnGA.

### Example 5

### Continuous Production of Glucose from Granular Cassava Starch by HgGA at a Neutral pH

The capability of HgGA to convert granular unmodified cassava starch to glucose and short chain glucose polymers at a neutral pH was further characterized. A 27% dry substance aqueous slurry of cassava starch was first adjusted to pH 6.4 with sodium carbonate. SPEZYME™ Alpha (Danisco US Inc., Genencor Division) was added at 2 AAU/g ds, and HgGA was added at 1 GAU/g ds. The reaction was carried out for 48 hours at 58° C. with continuous stirring. At selected time intervals, samples of the slurry were removed. The removed sample was added to a 2.5 ml micro-centrifuge tube and centrifuged for 4 minutes at 13,000 rpm. Refractive index (RI) of the supernatant was determined at 30° C. The remaining supernatant was filtered through a 13 mm syringe filter with a 0.45 μm GHP membrane into a 2.5 ml micro-centrifuge tube and boiled for 10 minutes to terminate the amylase activity. 0.5 mL enzyme-deactivated sample was diluted with 4.5 ml of RO water. The diluted sample was then filtered through 0.45 μm Whatman filters and subject to HPLC analysis. The HPLC analysis was conducted as described in Methods used in the Examples.

The total dry substance was determined by taking about 1 ml of the starch slurry into a 2.5 ml spin tube, adding 1 drop of SPEZYME® FRED (Danisco US Inc., Genencor Division) from a micro dispo-pipette, and boiling 10 minutes. Refractive index at 30° C. was determined. The dry substance of the supernatant and the whole sample (total) was determined using appropriate DE tables. The CRA 95 DE Table was used for the supernatant and corrected for consumption of water of hydrolysis. % soluble was calculated as: 100×(the dry substance of the supernatant)/(the total dry substance). The composition of the oligosaccharides is presented in Table 6.

TABLE 6
Saccharide distribution for HgGA-mediated saccharification of
cassava granular starch.

Saccharide Distribution

| hrs | DP1 | DP2 | DP3 | DP4+ | Soluble % |
|---|---|---|---|---|---|
| 2.50 | 93.799 | 1.726 | 0.499 | 3.976 | 56.20 |
| 7.50 | 96.166 | 1.551 | 0.480 | 1.802 | 78.80 |
| 12.00 | 96.731 | 1.639 | 0.411 | 1.220 | 85.10 |
| 23.50 | 96.928 | 2.204 | 0.326 | 0.541 | 92.80 |
| 48.00 | 96.772 | 3.023 | 0.205 | 0.000 | 99.00 |

As shown in Table 6, the reaction achieved about 93% solubility and yielded about 96.9% glucose within 24 hours. Continuation of saccharification resulted in 99% solubility and about 96.8% glucose after 48 hours.

### Example 6

### Continuous Production of Glucose from Granular Cornstarch by HgGA at a Neutral pH

Corn granular starch was used to characterize HgGA. The experiments were carried out using 32% ds corn granular starch. Water (64.44 g) and starch (35.56 g; at 90% ds) were mixed and the pH of the slurry was increased to 6.4. The starch slurry was placed in a water bath maintained at 58° C. and enzymes were added. The enzymes included SPEZYME™ Alpha (Danisco US Inc., Genencor Division) and HgGA. The starch slurry was maintained at 58° C. for 48 hrs and samples were drawn at 3, 6, 10, 24, 32, and 52 hrs to analyze the % soluble and saccharide profile. The results are presented in Table 7.

TABLE 7
Saccharide distribution for HgGA-mediated
saccharification of corn granular starch

| HgGA | Alpha-amylase | | | | | |
|---|---|---|---|---|---|---|
| (GAU/g ds) | (AAU/g ds) | hour | % Soluble | DP1 | DP2 | DP3+ |
| 1 | 2 | 3 | 56.82 | 94.74 | 1.57 | 3.69 |
| | | 6 | 69.45 | 95.52 | 1.76 | 2.61 |
| | | 10 | 75.96 | 96.50 | 1.79 | 1.43 |
| | | 24 | 91.50 | 95.72 | 2.79 | 0.93 |
| | | 32 | 92.71 | 95.50 | 3.08 | 0.86 |
| | | 52 | 99.66 | 93.94 | 4.42 | 0.67 |
| 0.75 | 2 | 3 | 53.35 | 92.74 | 2.00 | 5.25 |
| | | 6 | 65.87 | 94.69 | 1.77 | 3.43 |
| | | 10 | 73.11 | 95.80 | 1.73 | 2.12 |
| | | 24 | 89.09 | 95.70 | 2.53 | 1.59 |
| | | 32 | 91.01 | 95.75 | 2.64 | 1.01 |
| | | 52 | 98.65 | 95.44 | 3.44 | 1.12 |
| 0.5 | 2 | 3 | 49.06 | 88.36 | 3.36 | 8.29 |
| | | 6 | 61.98 | 92.48 | 2.18 | 5.35 |
| | | 10 | 68.18 | 94.08 | 1.90 | 3.67 |
| | | 24 | 84.14 | 95.56 | 2.03 | 2.23 |
| | | 32 | 87.90 | 95.49 | 2.25 | 2.11 |
| | | 52 | 95.17 | 95.30 | 2.81 | 1.12 |
| 0.25 | 2 | 3 | 44.01 | 75.08 | 9.16 | 15.76 |
| | | 6 | 53.92 | 84.31 | 5.25 | 10.45 |
| | | 10 | 60.97 | 88.25 | 3.72 | 7.81 |
| | | 24 | 76.63 | 93.11 | 2.25 | 4.48 |
| | | 32 | 80.00 | 93.66 | 2.17 | 4.05 |
| | | 52 | 88.37 | 94.55 | 2.31 | 2.89 |

As shown in Table 7, HgGA maintains a significant amount of glucoamylase activity for 52 hrs at pH 6.4, evidenced by the continued production of DP1 and DP2, as well as the continued increase of % soluble solids. The data also suggest that the rates of DP1 production and % solubilization of granular starch depend on the amount of HgGA. An increased amount of HgGA resulted in increased rates of % solubilization and DP1 production.

### Example 7

### Characterization of Granular Starch Hydrolysis by HgGA and SPEZYME™ Alpha at a Neutral pH by Scanning Electron Microscopy

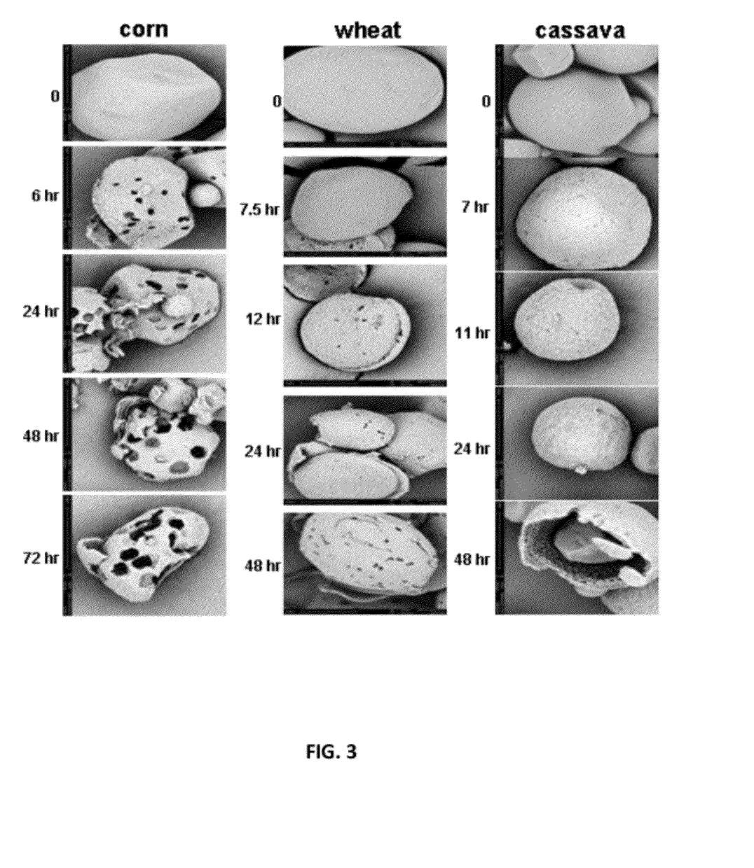

Granular starch from corn, wheat, and cassava was treated with HgGA and SPEZYME™ Alpha. A 28% dry substance aqueous slurry of granular starch was first adjusted to pH 6.4 with sodium carbonate. SPEZYME™ Alpha (Danisco US Inc., Genencor Division) was added at 2 AAU/g ds, and HgGA was added at 1 GAU/g ds. Treatment was carried out at 58° C. with continuous stirring. Samples of the slurry were removed at various time points and subject to scanning electron microscopy (SEM). Slurry samples were laid on SEM sample stubs using double-sided carbon tape. Excess sample was removed by gently dusting the mounted sample with compressed air. Mounted samples were sputter coated with gold (15 nm) for 2 min at 25 mV, using an Emitech K550 Sputter Coater (Squorum Technologies). The scanning electron micrographs are presented in FIG. 3. Before treatment, starch surface was smooth and homogenous. Upon HgGA and SPEZYME™ Alpha treatment, the surface morphology of the granules changed over time. The enzyme blend first created small dimples (0.2-0.5 μm in diameter) on the surface of the starch granules. Quantity and size of the dimples increased over time. At a late stage of the treatment, for example, 48 hours for cassava granular starch, empty shells were spotted. Micrographs of empty shells indicated a complete digestion of the interior of the granule. The mechanism of enzymatic action appears to be starch granule surface peeling. Once the surface has been weakened by external peeling, the amylases penetrate and hydrolyze the interior of the granule (i.e., amylolysis) leaving hollowed out shells.

### Example 8

### Lactic Acid Fermentation Using Various Glucoamylases with the SSF Process

Glucoamylases from various sources were tested for their use in lactic acid fermentation under a neutral pH. Lactic acid fermentation was carried out using SSF process. The substrate was 15% ds of cassava starch. Liquefaction was carried out at 85° C. for 90 min with the alpha-amylase GC 358 (Danisco US Inc., Genencor Division) dosed at 0.25 kg/ton. For saccharification and fermentation, 0.3 GAU/g HgGA, 0.3 GAU/g OPTIDEX™ L-400 Aspergillus niger glucoamylase (AnGA) (Danisco US Inc., Genencor Division), and 0.3 GAU/g of TrGA was used. The SSF were carried out at pH of 6.5 at 40° C., using inoculum of Lactobacillus rhamnosus. Samples were withdrawn at various time points for HPLC analysis, and the results are presented in Table 8.

TABLE 8
Lactic acid production during SSF

| | | | | | | | succinic | lactic | | acetic | | lactic |
|---|---|---|---|---|---|---|---|---|---|---|---|---|
| | | DP >3 | DP-3 | DP-2 | Glucose | Fructose | acid | acid | glycerol | acid | ethanol | acid |
| | time(h) | w/v % | w/v % | w/v % | w/v % | % w/v | % w/v | mg/mL | w/v % | w/v % | w/v % | g |
| AnGA | 4 | 3.80 | 0.67 | 0.00 | 6.40 | 0.05 | 0.11 | 9.71 | 0.00 | 0.08 | 0.08 | 10.68 |
| | 21 | 3.36 | 0.00 | 0.00 | 1.23 | 0.00 | 0.08 | 78.62 | 0.00 | 0.00 | 0.16 | 95.13 |
| | 45 | 2.83 | 0.00 | 0.17 | 0.19 | 0.00 | 0.09 | 106.41 | 0.00 | 0.00 | 0.18 | 130.89 |
| TrGA | 4 | 0.31 | 0.00 | 0.29 | 0.99 | 0.00 | 0.00 | 9.10 | 0.00 | 0.03 | 0.00 | 10.01 |
| | 21 | 0.15 | 0.03 | 0.04 | 0.32 | 0.00 | 0.00 | 88.73 | 0.00 | 0.04 | 0.04 | 107.36 |
| | 45 | 0.06 | 0.02 | 0.01 | 0.07 | 0.01 | 0.00 | 116.97 | 0.00 | 0.03 | 0.06 | 143.88 |
| HgGA | 4 | 3.75 | 0.65 | 0.00 | 6.94 | 0.00 | 0.11 | 9.64 | 0.00 | 0.08 | 0.06 | 10.60 |
| | 21 | 2.75 | 0.00 | 0.00 | 1.19 | 0.00 | 0.09 | 79.92 | 0.00 | 0.00 | 0.18 | 96.70 |
| | 45 | 1.51 | 0.43 | 0.56 | 0.16 | 0.00 | 0.09 | 108.53 | 0.00 | 0.00 | 0.23 | 133.49 |

Data presented in Table 8 indicates that when same amount of glucoamylases were used, both HgGA and TrGA resulted in more lactic acid production than AnGA at a neutral pH.

### Example 9

### Effect of Alpha-Amylase on Lactic Acid Fermentation in No-Cook Process

Various alpha-amylases were combined with TrGA to produce lactic acid from cornstarch through a no-cook process. The lactic acid fermentation was performed with 2 GAU/g or 1 GAU/g TrGA glucoamylase, combined with various alpha-amylases, fungal alpha-amylase GC 626, themostable bacterial alpha amylase SPEZYME® XTRA, and bacterial amylase AmyE (all from Danisco US Inc., Genencor Division). Each alpha-amylase was dosed at 1 kg/MT. Raw cornstarch was used as the substrate at 15% DS. Fermentation was carried out at 40° C. for 45 hrs using inoculum of Lactobacillus rhamnosus. Samples were withdrawn at various time points for HPLC analysis, and the results are presented in Table 9.

TABLE 9
Lactic acid production during SSF

| | | | | | | | succinic | lactic | | acetic | | lactic |
|---|---|---|---|---|---|---|---|---|---|---|---|---|
| | | DP >3 | DP-3 | DP-2 | Glucose | Fructose | acid | acid | glycerol | acid | ethanol | acid |
| | time(h) | w/v % | w/v % | w/v % | w/v % | % w/v | % w/v | mg/mL | w/v % | w/v % | w/v % | g |
| 1 kg/MT | 3.5 | 0.41 | 0.00 | 0.00 | 2.01 | 0.00 | 0.01 | 6.95 | 0.00 | 0.00 | 0.00 | 7.65 |
| GC626 | 21 | 0.30 | 0.00 | 0.04 | 1.19 | 0.01 | 0.04 | 33.43 | 0.00 | 0.21 | 2.11 | 38.44 |
| | 28 | 0.38 | 0.00 | 0.00 | 0.00 | 0.06 | 0.09 | 39.00 | 0.00 | 0.25 | 2.48 | 45.24 |
| | 45.5 | 0.42 | 0.00 | 0.00 | 0.00 | 0.08 | 0.18 | 43.67 | 0.00 | 0.31 | 2.59 | 51.53 |
| 1 kg/MT | 3.5 | 9.31 | 0.00 | 1.25 | 7.64 | 0.35 | 0.21 | 9.24 | 0.00 | 0.38 | 0.29 | 10.17 |
| SPEZYME ® | 21 | 8.50 | 0.00 | 0.00 | 0.04 | 0.25 | 0.30 | 39.61 | 0.07 | 3.00 | 1.77 | 45.94 |
| XTRA | 28 | 11.91 | 0.00 | 0.06 | 0.05 | 0.15 | 0.30 | 47.91 | 0.10 | 3.92 | 5.40 | 56.54 |
| | 45.5 | 12.33 | 0.00 | 0.05 | 0.03 | 0.13 | 0.26 | 49.34 | 0.00 | 3.61 | 6.76 | 59.71 |
| 1 kb/MT | 3.5 | 0.30 | 0.00 | 0.17 | 0.55 | 0.07 | 0.03 | 9.13 | 0.00 | 0.02 | 0.00 | |
| AmyE | 21 | 0.29 | 0.07 | 0.03 | 0.01 | 0.03 | 0.04 | 44.05 | 0.00 | 0.15 | 0.10 | 10.04 |
| | 28 | 0.29 | 0.12 | 0.02 | 0.00 | 0.02 | 0.03 | 55.46 | 0.00 | 0.15 | 0.10 | 51.54 |
| | 45.5 | 0.37 | 0.13 | 0.02 | 0.03 | 0.01 | 0.04 | 72.91 | 0.00 | 0.28 | 0.21 | 66.00 |

Data presented in Table 9 indicate that AmyE combined with TrGA resulted in the highest lactic acid yield, while fungal alpha-amylase GC 626 resulted in the lowest lactic acid yield.

Furthermore, the lactic acid fermentation was further performed using 1 kg/t AmyE with 1 GAU/g of HgGA, AnGA, or TrGA with the no-cook process. The fermentation was performed at pH 6.5, 40° C., with 15% DS of cornstarch. Samples were withdrawn at various time points for HPLC analysis, and the results are presented in Table 10.

TABLE 10
Lactic acid production during SSF

| | | | | | | | succinic | lactic | | acetic | |
|---|---|---|---|---|---|---|---|---|---|---|---|
| | | DP >3 | DP-3 | DP-2 | Glucose | Fructose | acid | acid | glycerol | acid | ethanol |
| | time(h) | w/v % | w/v % | w/v % | w/v % | % w/v | % w/v | mg/mL | w/v % | w/v % | w/v % |
| HgGA | 5 | 0.32 | 0.00 | 0.04 | 1.06 | 0.11 | 0.05 | 17.96 | 0.00 | 0.04 | 0.02 |
| | 22 | 0.43 | 0.10 | 0.02 | 0.02 | 0.02 | 0.04 | 60.90 | 0.00 | 0.15 | 0.14 |
| | 30 | 0.37 | 0.12 | 0.03 | 0.05 | 0.06 | 0.05 | 74.03 | 0.00 | 0.17 | 0.19 |
| | 51 | 0.45 | 0.17 | 0.03 | 0.02 | 0.02 | 0.06 | 104.74 | 0.00 | 0.28 | 0.35 |
| TrGA | 5 | 0.03 | 0.00 | 0.02 | 0.06 | 0.01 | 0.00 | 9.13 | 0.00 | 0.04 | 0.00 |
| | 21 | 0.03 | 0.01 | 0.00 | 0.00 | 0.00 | 0.00 | 44.05 | 0.00 | 0.15 | 0.01 |
| | 29 | 0.04 | 0.01 | 0.00 | 0.00 | 0.00 | 0.00 | 55.46 | 0.00 | 0.17 | 0.01 |
| | 52 | 0.04 | 0.01 | 0.00 | 0.00 | 0.00 | 0.00 | 76.40 | 0.00 | 0.28 | 0.03 |
| AnGA | 5 | 0.03 | 0.00 | 0.01 | 0.04 | 0.01 | 0.01 | 9.82 | 0.00 | 0.04 | 0.00 |
| | 21 | 0.03 | 0.01 | 0.00 | 0.00 | 0.00 | 0.00 | 34.26 | 0.00 | 0.15 | 0.02 |
| | 29 | 0.03 | 0.01 | 0.00 | 0.00 | 0.00 | 0.00 | 34.96 | 0.00 | 0.17 | 0.03 |
| | 52 | 0.03 | 0.01 | 0.00 | 0.00 | 0.00 | 0.00 | 24.52 | 0.00 | 0.28 | 0.32 |

Data presented in Table 10 indicate HgGA combined with AmyE resulted in the highest lactic acid yield, while AnGA combined with AmyE resulted in the lowest lactic acid yield.

### Example 10

### Comparison of Various Fermentation Processes—Glucose as Substrate Vs. Conventional Full Saccharification Process from Starch

Glucose or the glucose syrup resulting from conventional liquefaction and full saccharification of cassava starch was used as the substrate for lactic acid fermentation. When glucose was used as the substrate, the inoculum of Lactobacillus rhamnosus was transferred to each 100 mL seed culture and cultivated at 37° C., 200 rpm. Then 10 mL seed culture was added to each fermentor with 1 L fermentation medium. The fermentation temperature was controlled at 40° C. For the glucose syrup from conventional full saccharification of cassava starch, liquefaction was carried at 85° C. for 90 min with 15% DS cassava starch, using alpha-amylase GC358 (Danisco US Inc., Genencor Division) at a dose of 0.25 kg/ton. When the mash was cool down to 60° C., saccharification was performed for about 18 h with 1 kg/ton AnGA. Fermentation was performed as using 15% glucose. Samples were withdrawn at various time points for HPLC analysis, and the results are presented in Table 11.

TABLE 11
Comparison of lactic acid fermentation using (1) glucose and (2) the conventional full saccharification product of
cassava starch

| | | | | | | | succinic | lactic | | acetic | |
|---|---|---|---|---|---|---|---|---|---|---|---|
| | | DP >3 | DP-3 | DP-2 | Glucose | Fructose | acid | acid | glycerol | acid | ethanol |
| | time(h) | w/v % | w/v % | w/v % | w/v % | % w/v | % w/v | mg/mL | w/v % | w/v % | w/v % |
| glucose | 4 | 0.21 | 0.00 | 0.05 | 11.72 | 0.00 | 0.00 | 6.26 | 0.00 | 0.00 | 0.00 |
| | 21 | 0.28 | 0.11 | 0.07 | 1.85 | 0.00 | 0.04 | 74.73 | 0.00 | 0.07 | 0.19 |
| | 24 | 0.28 | 0.13 | 0.08 | 0.43 | 0.05 | 0.02 | 83.61 | 0.00 | 0.07 | 0.20 |
| | 28 | 0.28 | 0.12 | 0.07 | 0.01 | 0.05 | 0.03 | 81.92 | 0.00 | 0.08 | 0.25 |
| | 46 | 0.26 | 0.12 | 0.07 | 0.01 | 0.05 | 0.04 | 85.54 | 0.00 | 0.18 | 0.23 |
| fully | 4 | 0.81 | 0.15 | 0.33 | 15.45 | 0.00 | 0.02 | 8.65 | 0.00 | 0.03 | 0.00 |
| saccharified | 21 | 0.92 | 0.26 | 0.31 | 5.14 | 0.00 | 0.03 | 82.61 | 0.00 | 0.03 | 0.05 |
| cassava | 31 | 0.96 | 0.27 | 0.31 | 3.82 | 0.01 | 0.04 | 95.12 | 0.00 | 0.04 | 0.06 |
| starch | 45 | 0.91 | 0.26 | 0.29 | 1.10 | 0.07 | 0.04 | 111.70 | 0.00 | 0.05 | 0.07 |

Data presented in Table 8 indicate that there were still fermentable sugars remaining (1.10%) after 46 hours in the fermentation using conventional full saccharification product, while the glucose level dropped to 0.01% in the fermentation directly using glucose. Furthermore, compared with the fermentation directly using glucose, the fermentation using conventional full saccharification product resulted in a greater lactic acid yield and less amount of impurities (i.e., glycerol, acetic acid, and ethanol).

### Example 11

### Comparison of Various Fermentation Processes—SSF Vs. Glucose Syrup from Direct Granular Starch Conversion

Three different processes using 15% DS cassava starch as the substrate for lactic acid fermentation were investigated. For SSF process, pH was adjusted to 6.5, liquefaction was carried at 85° C. for 90 minutes using 15% DS cassava starch, and alpha-amylase GC358 (Danisco US Inc., Genencor Division) was dosed at 0.25 kg/t. 0.3 GAU/g TrGA was applied when the mash was cool down to 40° C. for SSF. For the no-cook full saccharification process, pH was adjusted to 6.5, and 15% DS cassava starch was supplemented with 1.0 GAU/g HgGA and 0.5 kg/MT SPEZYME® XTRA (Danisco US Inc., Genencor Division). The saccharification was performed at 60° C. for 18 hrs before fermentation. For the no-cook process, the slurry was adjusted to pH 6.5, supplemented with 2.0 GAU/g HgGA and 1.0 kg/MT AmyE, and then subject to fermentation directly. The inoculum of Lactobacillus rhamnosus was transferred to each 100 mL seed culture and cultivated at 37° C., 200 rpm. Then 10 mL seed culture was added to each fermentor with 1 L fermentation medium. The fermentation temperature was controlled at 40° C. Samples were withdrawn at various time points for HPLC analysis, and the results are presented in Table 12.

TABLE 12
Comparison of lactic acid fermentation using (1) SSF, (2) no-cook full saccharification fermentation, and (2) no-
cook direct fermentation

| | | | | | | | succinic | lactic | | acetic | |
|---|---|---|---|---|---|---|---|---|---|---|---|
| | | DP >3 | DP-3 | DP-2 | Glucose | Fructose | acid | acid | glycerol | acid | ethanol |
| | time(h) | w/v % | w/v % | w/v % | w/v % | % w/v | % w/v | mg/mL | w/v % | w/v % | w/v % |
| SSF | 4 | 0.31 | 0.00 | 0.29 | 0.99 | 0.00 | 0.00 | 9.10 | 0.00 | 0.03 | 0.00 |
| | 21 | 0.15 | 0.03 | 0.04 | 0.32 | 0.00 | 0.00 | 88.73 | 0.00 | 0.04 | 0.04 |
| | 31 | 0.12 | 0.03 | 0.02 | 0.25 | 0.00 | 0.00 | 98.54 | 0.00 | 0.04 | 0.05 |
| | 45 | 0.06 | 0.02 | 0.01 | 0.07 | 0.01 | 0.00 | 116.97 | 0.00 | 0.03 | 0.06 |
| No-cook full | 0 | 1.41 | 0.00 | 1.16 | 11.21 | 0.00 | 0.00 | 0.00 | 0.00 | 0.02 | 0.00 |
| saccharification | 5 | 1.71 | 0.00 | 1.07 | 10.65 | 0.00 | 0.01 | 6.98 | 0.00 | 0.03 | 0.00 |
| fermentation | 21 | 1.20 | 0.38 | 0.52 | 0.09 | 0.09 | 0.05 | 89.66 | 0.00 | 0.00 | 0.04 |
| | 28 | 0.74 | 0.32 | 0.30 | 0.11 | 0.01 | 0.01 | 92.91 | 0.00 | 0.00 | 0.00 |
| | 52 | 0.96 | 0.38 | 0.13 | 0.01 | 0.00 | 0.03 | 96.28 | 0.00 | 0.00 | 0.05 |
| No-cook direct | 5 | 0.32 | 0.00 | 0.01 | 3.11 | 0.11 | 0.04 | 10.48 | 0.00 | 0.03 | 0.00 |
| fermentation | 22 | 0.44 | 0.11 | 0.03 | 0.03 | 0.05 | 0.06 | 74.87 | 0.00 | 0.08 | 0.13 |
| | 30 | 0.43 | 0.11 | 0.04 | 0.05 | 0.05 | 0.07 | 91.35 | 0.00 | 0.08 | 0.15 |
| | 51 | 0.47 | 0.12 | 0.02 | 0.00 | 0.02 | 0.05 | 116.37 | 0.00 | 0.11 | 0.30 |

Results presented in Table 9 indicate that the lactic acid yield was similar among all three processes. Traditionally liquefied cassava starch resulted in 116.97 mg/ml of lactic acid at 45 hours. For the cassava starch subject to the no-cook process, direct fermentation without saccharification resulted in 116.37 mg/ml of lactic acid at 51 hours, while fermentation of the fully saccharified substrate resulted in 96.28 mg/ml of lactic acid at 52 hours. The results further suggest that the no-cook process starting with raw starch may save at least 18 hours that was spent for saccharification.

### Example 12

### Fermentation Using Starch Substrates with a High Dry Solid (DS) Value

The granular cornstarch slurry having a DS of 18%, 20%, or 25% was adjusted to pH 6.5, supplemented with 2.0 GAU/g HgGA and 1.0 kg/MT AmyE, and then subject to fermentation directly. For the fermentation, the inoculum of Lactobacillus rhamnosus was transferred to each 100 mL seed culture and cultivated at 37° C., 200 rpm. Then 10 mL seed culture was added to each fermentor with 1 L fermentation medium. The fermentation temperature was controlled at 40° C. Samples were withdrawn at various time points for HPLC analysis, and the results are presented in Table 13.

TABLE 13
Lactic acid production using 18%, 20% and 25% DS cornstarch.

| | | Glucose | lactic acid | Productivity |
|---|---|---|---|---|
| DS | Time(h) | % w/v | mg/mL | (g/L · h) |
| 18% | 5 | 4.74 | 8.75 | 1.75 |
| | 20 | 0.20 | 83.41 | 4.28 |
| | 28 | 0.13 | 103.62 | 3.70 |
| | 45 | 0.02 | 118.25 | 2.63 |
| | 53 | 0.02 | 122.89 | 2.34 |
| | 69 | 0.01 | 128.01 | 1.86 |
| 20% | 4 | 4.64 | 7.31 | 1.83 |
| | 22 | 0.13 | 89.27 | 4.15 |
| | 29 | 0.20 | 109.15 | 3.76 |
| | 45 | 0.05 | 125.88 | 2.80 |
| | 53 | 0.24 | 137.60 | 2.60 |
| | 117 | 0.05 | 144.36 | 1.23 |
| 25% | 4 | 3.58 | 7.71 | 1.93 |
| | 22 | 0.24 | 102.40 | 4.76 |
| | 29 | 0.48 | 118.89 | 4.10 |
| | 45 | 1.16 | 137.15 | 3.05 |
| | 53 | 4.01 | 151.25 | 2.85 |
| | 117 | 0.53 | 164.94 | 1.41 |

The data presented in Table 13 indicate that HgGA combined with AmyE could be used to ferment lactic acid directly from granular starch having a high DS. This observation further suggests an increase of plant capacity without additional capital cost. As shown in Table 13, even at a high DS (25% ds), the rate of lactic acid production reached 4.76 g/L per hour, at 22 hrs.

### Example 13

### Fermentation Under Various pH Conditions

The no-cook direct fermentation process was further performed at pH 6.5 and pH 7.0. The slurry was adjusted to pH 6.5 or pH 7.0, supplemented with 1.5 GAU/g TrGA and 1.0 kg/MT SPEZYME® XTRA, and then subject to fermentation directly. The inoculum of Lactobacillus rhamnosus was transferred to each 100 mL seed culture and cultivated at 37° C., 200 rpm. Then 10 mL seed culture was added to each fermentor with 1 L fermentation medium. The fermentation temperature was controlled at 40° C. Samples were withdrawn at various time points for HPLC analysis, and the results are presented in Table 14.

TABLE 14
Lactic acid production at pH 6.5 and pH 7.0.

| | | | | | | | succinic | lactic | acetic | |
|---|---|---|---|---|---|---|---|---|---|---|
| | | DP >3 | DP-3 | DP-2 | Glucose | Fructose | acid | acid | acid | ethanol |
| | time(h) | w/v % | w/v % | w/v % | w/v % | % w/v | % w/v | mg/mL | w/v % | w/v % |
| pH | 4 | 0.73 | 0.00 | 0.17 | 4.20 | 0.03 | 0.03 | 7.98 | 0.02 | 0.00 |
| 6.5 | 22 | 1.01 | 0.32 | 0.27 | 0.52 | 0.08 | 0.07 | 87.71 | 0.03 | 0.00 |
| | 29 | 0.74 | 0.31 | 0.13 | 0.83 | 0.08 | 0.08 | 97.57 | 0.03 | 0.00 |
| | 45 | 0.60 | 0.27 | 0.11 | 0.36 | 0.00 | 0.08 | 112.13 | 0.03 | 0.03 |
| | 53 | 0.52 | 0.25 | 0.10 | 0.28 | 0.00 | 0.09 | 117.45 | 0.03 | 0.03 |
| | 70 | 0.43 | 0.23 | 0.08 | 0.24 | 0.00 | 0.08 | 120.23 | 0.03 | 0.00 |
| pH | 5 | 0.77 | 0.00 | 0.49 | 3.11 | 0.01 | 0.02 | 8.42 | 0.02 | 0.00 |
| 7.0 | 22 | 0.81 | 0.29 | 0.06 | 0.34 | 0.03 | 0.06 | 91.60 | 0.04 | 0.04 |
| | 28 | 0.73 | 0.28 | 0.04 | 0.17 | 0.01 | 0.03 | 103.02 | 0.03 | 0.04 |
| | 45 | 0.50 | 0.24 | 0.04 | 0.24 | 0.02 | 0.06 | 118.11 | 0.02 | 0.04 |
| | 52 | 0.38 | 0.18 | 0.03 | 0.39 | 0.02 | 0.06 | 120.56 | 0.02 | 0.04 |
| | 69 | 0.36 | 0.19 | 0.03 | 0.24 | 0.02 | 0.06 | 124.86 | 0.04 | 0.04 |

As shown in Table 14, the lactic acid production results over time were similar at pH 6.5 and pH 7.0. The data suggest that the operation pH for the no-cook direct fermentation can be flexible, for example, in the range of 6.5 to 7.0.

### Example 14

### No-Cook Process Adapted to Glucose-Based Strains

Two lactic acid-producing microorganism, Bacillus coagulans and Lactobacillus rhamnosus, were used ferment lactic acid with the no-cook process. The cornstarch slurry was adjusted to pH 6.5, supplemented with 1 GAU/g HgGA and 1.0 kg/MT AmyE, and then subject to fermentation directly. The inoculum of Bacillus coagulans or Lactobacillus rhamnosus was transferred to each 100 mL seed culture and cultivated at 37° C., 200 rpm. Then 10 mL seed culture was added to each fermentor with 1 L fermentation medium. The fermentation temperature was controlled at 40° C. Fermentation was also performed as using 15% glucose. Samples were withdrawn at various time points for HPLC analysis, and the results are presented in Table 15.

TABLE 15
Lactic acid production by different microorganism using
different feed stocks

| | | | | lactic | |
|---|---|---|---|---|---|
| | Substrate | | Glucose | acid | Productivity |
| Microorganism | Process | Time(h) | % w/v | mg/mL | (g/L · h) |
| Bacillus | 15% glucose | 5 | 13.89 | 7.38 | 1.48 |
| coagulans | convention | 22 | 11.22 | 19.19 | 0.87 |
| | process | 29 | 6.72 | 48.47 | 1.67 |
| | Cornstarch | 5 | 3.33 | 8.29 | 1.66 |
| | No-cook | 22 | 1.90 | 38.80 | 1.76 |
| | | 29 | 1.08 | 53.93 | 1.86 |
| Lactobacillus | 15% glucose | 4 | 11.72 | 6.16 | 1.76 |
| rhamnosus | convention | 21 | 1.85 | 74.63 | 3.55 |
| | process | 28 | 0.01 | 81.81 | 2.92 |
| | Cornstarch | 3 | 1.69 | 8.67 | 3.47 |
| | No-cook | 19 | 0.40 | 78.44 | 4.13 |
| | | 27 | 0.20 | 98.49 | 3.65 |

The data presented in Table 15 indicate that no-cook process resulted in (1) a higher lactic acid yield, and (2) a lower or at least equivalent level of residual glucose, for both Bacillus coagulans and Lactobacillus rhamnosu. This observation suggests that the no-cook process could adapt to glucose-based strains.

### Example 15

### Succinic Acid Fermentation Using HgGA

The following is a prophetic example. This experiment will be carried out in 1 L bioreactor to monitor succinic acid formation from granular starch using enzymes with glucoamylase activity mentioned in Example 5, at desired fermentation conditions of pH 6.7 and temperature 34° C. For this experiment, raw granular starch in slurry form (maximum final concentration 80 g/L glucose) in 0.5×TM2 fermentation medium, will be pasteurized (i.e. the mixture held at 34° C. for 30 min for germination of any contaminant present in the starch slurry, and then pasteurized at 65′C for 14 hr). The pasteurized starch will be added to the pre-sterilized 1 L bioreactor. The pH of the starch slurry plus medium will be adjusted to 6.7 and controlled at 6.65 with NH₄OH. Then, the desired enzymes mentioned in Example 6 will be added as 0.2 micron filtered solution (20 ml) in DI water. An inoculum of succinic acid-producing strain 36 1.6 ppc E. coli, taken from frozen vial, will be prepared in TM2+10 g/L glucose medium. After the inoculum grows to OD 3-4, measured at 550 nm, 70 ml will be added to the bioreactor. At 3.7 hours into the run, the air being sparged at 0.6 slpm will be switched to nitrogen gas at 0.6 slpm. During the fermentation, samples will be taken from the vessel, centrifuged and the supernatants will be refrigerated to terminate the enzyme action. The supernatants will be subjected to HPLC analysis to estimate the bioconversion of granular starch by measuring glucose formation and its conversion to succinate at 34° C. and pH 6.7.

### Example 16

### 1,3-propanediol Fermentation Using HgGA

The following is a prophetic example. This experiment will be carried out in 1 L fermentor to monitor 1,3-propanediol formation from granular starch using enzymes with glucoamylase activity at the desired fermentation pH 6.7 and temperature 34° C. For this experiment, granular starch in slurry form (for maximum final concentration 100 g/L glucose) in 0.5×TM2 fermentation medium will be pasteurized (the slurry mixture held at 34° C. for 30 min for germination of any contaminant present in the starch slurry, and then inactivated at 65° C. for 14 hr). Then, pasteurized starch will be added to the pre-sterilized 1 L fermentor. The pH of the slurry plus fermentation medium will be adjusted to 6.7 and controlled at 6.65 with NH₄OH. Then, the desired enzyme activity and requirements specific for 1,3-propanediol production (30 mg spectinomycin and 2 mg vitamin B12) will be added in DI water. An inoculum of 1,3-propanediol-producing E. coli strain TTaldABml/p109F1 taken from a frozen vial, will be prepared in soytone-yeast extract-glucose medium. After the inoculum grows to OD 3-4 (measured at 550 nm), 70 ml will be transferred to the 1 L fermentor. During the fermentation, samples will be taken from the fermentor, centrifuged, and supernatants will be subjected to HPLC analysis. This will determine the fermentative bioconversion of granular starch by measuring glucose formation and its conversion to glycerol (1,3-propanediol pathway intermediate) and then to 1,3-propanediol.

What is claimed is:

1. A method of processing starch comprising saccharifying a starch substrate to fermentable sugars at pH 5.0 to 8.0 in the presence of a glucoamylase, wherein the glucoamylase possesses at least 50% activity at pH 6.0 or above relative to its maximum activity, wherein the glucoamylase is selected from the group consisting of Humicola grisea glucoamylase (HgGA) comprising SEQ ID NO: 3, Trichoderma reesei glucoamylase (TrGA) comprising SEQ ID NO: 6, Rhizopus sp. glucoamylase (RhGA) comprising SEQ ID NO: 9, and a variant thereof, and wherein the variant has at least 99% sequence identity to a parent glucoamylase.

2. The method of claim 1, wherein the variant has one amino acid modification compared to the parent glucoamylase.

3. The method of claim 1, wherein the HgGA is SEQ ID NO: 3.

4. The method of claim 3, wherein the HgGA is produced from a Trichoderma reesei host cell.

5. The method of claim 1, wherein the TrGA is SEQ ID No: 6.

6. The method of claim 1, wherein the RhGA is SEQ ID NO: 9.

7. The method of claim 1, wherein saccharifying is carried out at a pH in a range of 6.0 to 7.5.

8. The method of claim 1, wherein saccharifying is carried out at a pH in a range of 7.0 to 7.5.

9. The method of claim 1 further comprising fermenting the fermentable sugars to an end product, and wherein saccharifying and fermenting are performed at the same pH.

10. The method of claim 9, wherein saccharifying and fermenting are carried out as a simultaneous saccharification and fermentation (SSF) process.

11. The method of claim 10, wherein the SSF process is carried out at a pH between 7.0 to 7.5.

12. The method of claim 1, wherein saccharifying is performed at a temperature in a range of about 30° C. to about 60° C.

13. The method of claim 12, wherein saccharifying is performed at a temperature in a range of about 40° C. to about 60° C.

14. The method of claim 1, wherein the starch substrate is about 15% to 50% dry solid (DS).

15. The method of claim 1, wherein the starch substrate is about 15% to 30% dry solid (DS).

16. The method of claim 1, wherein the starch substrate is about 15% to 25% dry solid (DS).

17. The method of claim 9, wherein the end product is selected from the group consisting of methanol, ethanol, butanol, monosodium glutamate, succinic acid, 1,3-propanediol, vitamins, amino acids, and lactic acid.

18. The method of claim 17, wherein the end product is ethanol.

19. The method of claim 17, wherein the end product is 1,3-propanediol.

20. The method of claim 17, wherein the end product is succinic acid.

21. The method of claim 1, wherein the starch substrate is granular starch or liquefied starch.

22. The method of claim 1, wherein the glucoamylase is dosed at a range of about 0.1 to about 2.0 GAU per gram of dry substance starch.

23. The method of claim 22, wherein the glucoamylase is dosed at a range of about 0.2 to about 1.0 GAU per gram of dry substance starch.

24. The method of claim 22, wherein the glucoamylase is dosed at a range of about 0.5 to 1.0 GAU per gram of dry substance starch.

25. The method of claim 1 further comprising adding an alpha-amylase.

26. The method of claim 25, wherein the alpha-amylase is from a Bacillus species, or a variant thereof.

27. The method of claim 26, wherein the alpha-amylase is a Bacillus subtilis alpha-amylase (AmyE), a Bacillus amyloliquefaciens alpha-amylase, a Bacillus licheniformis alpha-amylase, a Bacillus stearothermophilus alpha-amylase, or a variant thereof.

28. The method of claim 1, wherein the starch substrate is from corn, wheat, rye, barley, sorghum, cassava, tapioca, potato and any combination thereof.

29. A method of processing starch comprising saccharifying a starch substrate to fermentable sugars at pH 5.0 to 8.0 in the presence of glucoamylase and at least one other enzyme,
wherein the glucoamylase possesses at least 50% activity at pH 6.0 or above relative to its maximum activity, wherein the glucoamylase is selected from the group consisting of Humicola grisea glucoamylase (HgGA) comprising SEQ ID NO: 3, Trichoderma reesei glucoamylase (TrGA) comprising SEQ ID NO: 6, Rhizopus sp. glucoamylase (RhGA) comprising SEQ ID NO: 9, and a variant thereof, and wherein the variant has at least 99% sequence identity to a parent glucoamylase, and
wherein the other enzyme is selected from the group consisting of proteases, pullulanases, isoamylases, cellulases, hemicellulases, xylanases, cyclodextrin glycotransferases, lipases, phytases, laccases, oxidases, esterases, cutinases, xylanases, and alpha-glucosidases.

30. A method of processing starch comprising saccharifying a starch substrate to fermentable sugars at pH 5.0 to 8.0 in the presence of glucoamylase and at least one other non-starch polysaccharide hydrolyzing enzymes,
wherein the glucoamylase possesses at least 50% activity at pH 6.0 or above relative to its maximum activity, wherein the glucoamylase is selected from the group consisting of Humicola grisea glucoamylase (HgGA) comprising SEQ ID NO: 3, Trichoderma reesei glucoamylase (TrGA) comprising SEQ ID NO: 6, Rhizopus sp. glucoamylase (RhGA) comprising SEQ ID NO: 9, and a variant thereof, and wherein the variant has at least 99% sequence identity to a parent glucoamylase, and
wherein the non-starch polysaccharide hydrolyzing enzymes is selected from the group consisting of cellulases, hemicellulases and pectinases.
